SEQUENCE LISTING (10 sequences)

SEQ ID NO: 1 - DNA, 3990 bp, Homo sapiens
tttcctacccccatttctcttcacaggtgtgcagagaagtctacattcccatgtcaagtaagtggggtgggtagatgggagggcatatgagagcggggcagcaattttatgtcttagttggaaggaagtttagagttcattgagtccaaatcctctttactaggtgaggaaactgaggcacaggagggggaggtgccattagccagggagtcagagccaggacttagaacctggtccatgcctcctagtttgttcctgaccaatcctcccagctcacccactccttttgctctcacctctgactccctacaggctacagctagagagttgactcctctatttgaggtaagagtgtctggatttgggataaggtgacctcagtttgcaggtctcatgggggagatcagagggtcctttgttgagaagaggggacttggtccacagtgagcaaaggaatttgtctaaattgctattaagggagatcatctcccaaggtgggccaagcctgaaccctcagccctccatttcttccctttcttttgccttcagattgacaggcctcggaagtcaaaataagtggtttcctagaccgggtcgagagcaagtctctattggtcccaactgagttttttcagctggtttttcaaccaaacagcacctcatctcccagtgaggggaagggaaggctgggctgagagcagcaaggctgctcatctcacctctccccacccagccatgccagccgccacacctggtggggagaggtgggcctcacctgggtcccctggcagtgctctgtgaagggtcttgacattgcactgtaataataaaggtgtgtgtgaagtatctttttatggtgactttctaaaacccagggaatcatgggaccagttctgatgactcagcctggcttccagtctcctccaggcccaacggtggcccccagcactggttggggcctgggagagctggccttggctgaagtgaagccacctacccttcaggcataacaggacagtgagaaggaaggaaagcctgcctcaacctcccatcagccctgagcaccccagaagggggccggctaggagtctaggcatgcaggaggctgacccctgactgggctcatatccagccacaaggcagccagggacccaggcacccaccccttgtctgcgtccctctcgggaatgggcctcttgcccaggccagaaatacaccacctacagtacaaattataatctaaaaacaagagggtggtgttgagtggggaaattggggaaggtgttttaggagccactaggaaaatgggcagcagggactctctggactggcttgggaagagcgcttttggggaacctggaggatggcaagctgagaaacactggtgtggagattccagccaaatcccaggcctgcccctccccctcctctgagaggccgtcttcttggcagacagcagagagatgcatgacaaaggtgccgtgatggttctgtcctggggattgagatggctggggaggggcctcctcctgttccgaagcatgttcctcccacccccaccaggccccataatctacctgccttttgggcagttaaaggccgagaagtgaacacagctgcaaccccactgccttgtagaccttccggcagacctgtggcaggtattgaaatgcacgcatacaattaggctcaaaaagtctacacagacaggagatgggcacacgaacagaggcaacataagagtgggggaaaagtctcaaaagactcacggatgccaccaagatgaagacagctggccacgggacacccatccccttagaaggcagacagagccactgaccccagcagacaagcccaggcagggctgagcctggagcctgcaatgagaagccttacttaagtcgacagaggtcagcgtgcccagtccagacctggccttctggccttcgaagctgtggggagccctggcccagagccccctctggagcccccagacttaccccaggccctccactgagatcaagttttgggagcagacagacaaacatcatccctcacagacaggcattccgttggctattctcttgcaaacagaatcaagcactagaccagcagcatgagcctcaggatactcaggccaggcccagaaaaacagaccctgaaggggagcttagggcagccttcctgcacccctccacaaatcactcgccacctcctctgcgtctttctgccagccagccccactaaacaaagcacatccctcaatcttccgggctcggggagggatgcacgatgaagctggacgcctgagtcccccagaggaaggaggaactagatacctaggtccctgtagggggcccttggtgcccgtctgaggctcagtctttgaggggattgcagaggggggttgctggagctccttttagcgtctctgaaggggattctgtgtgaggggattgggactggggggttggggagcaggaagcagtccccaggggagccatccaggcccattcaagggttgagcacttgtttagggttagagctgccccctctggggaccgggattgtccagccaaggccattgtcctgcccccttcccccagtccctcccaggcttctttgaacctgaagtcagatattttttctccacaccccccaccccctggttttccccacccagggcctagggctggaggcctgggccagggaggtgggggagggagaacggggcctaccgtggtattagatgtctgagttttggttgagaggggagcaaggaacctgatgtgcaggttccatagtggagggggcccaaagcgggtgtcttatcactctgtttcagcaaaggttgggaaactgaggcccaatcagtccaaagtctggtcccttgaaggggaagtaggggccaaccccttagtctgttagatgaggagagtctggagtctgattctggaagacggaggggtggggggatggggggtggggggatatagcacggaggccttgtctggcagtctactcttgaagatggggtgaaatttggcaggctgggcagatggtgccaggcacccaggctgcggggtggctggatttggccagtatcgggatgggaatgcctaggattctggatggatcgggggaaggcataagggagcagctggccattgtgcttatggctgttgatgcattgagggatagcgccacacacacattcaataaatttgaggagctgagagggtgactggcccctgaaggcacagtgccagaggtctgtggagagggggtcaagcacctgggttcctgaagaacatggaggtgtgggagtgattccagacagctgggatgtgcagagcctgagagagtgccagggagcgggttgggagttgaaagttgggtgtggtggctcacgcctttaatcatgacactgggcggcagaggcgggaggatttcttgaggacaggaattcaagaccagcctgggtaacatagcaaggccccatctctactaaaaataaaaaaactaacagggcacagtggtccaagcctgtagtcccagccacttaggaggctggagcagaaggattgctttggcccagtagatcgaggctacattgagccatcattgtactccactgcactccagtctgggcaacaaagtgagaccctgtcttaaaaaataaaaataaaaaaagtttctgtgggggacctgcactgaggtcctggaggggcgccagttgtgtctcccggttttccccttccacagacaccattgccaccaccattaggcaaacatccttcgcctcagtttctccccccacctccctctcctccacccatccagggggcggggccagaggtcaaggctagtgggtgggactggggagggagagaggggttgagtagtcccttcgcaagccctcatttcaccaggcccccggcttggggcgccttccttccccatggcgggacacctggcttcagattttgc

SEQ ID NO: 2 - DNA, 22 bp, Artificial Sequence
  Description of Artificial Sequence Synthetic primer
tggaacctgcacatcaggttcc

SEQ ID NO: 3 - DNA, 21 bp, Artificial Sequence
  Description of Artificial Sequence Synthetic primer
tcagtctttgaggggattgca

SEQ ID NO: 4 - DNA, 23 bp, Artificial Sequence
  Description of Artificial Sequence Synthetic primer
tcccagctgtctggaatcactcc

SEQ ID NO: 5 - DNA, 22 bp, Artificial Sequence
  Description of Artificial Sequence Synthetic primer
ctaccgtggtattagatgtctg

SEQ ID NO: 6 - DNA, 20 bp, Artificial Sequence
  Description of Artificial Sequence Synthetic primer
tagattatggggcctggtgg

SEQ ID NO: 7 - DNA, 21 bp, Artificial Sequence
  Description of Artificial Sequence Synthetic primer
ctaggagtctaggcatgcagg

SEQ ID NO: 8 - DNA, 1890 bp, Mus musculus
aagctttgtgaacttggcggcttccaagtcgctgcctttatttaggtcttccaactaactatggcactgttccacaatgaatgtatagaaattgggaggtgagcatgacagagtggaggaaacggaagattcatggagagggccagagagatggcccctcagccaccctgggggatgacttggacccatgtggtagaaggaggggactttcacacatgtgctatgtgtagctgtgtgtaggtacatacacacccttaaaataaaacgcaatttttttttcaaagtctcagggtgaatttggtgaagtcgatgaagctgaggcaggagaattatcaggagttcaagggcagcttgttttatagagaaaggttccatctctacctgatgaagactaccatcaagagacacccccgccccccagggcacctagagccactgaccctagccaacagctcaggcgggctgggcccaggctcagaactctgtcctggctatgtacactgtggggtgctctgggctttttgaggctgtgtgattcaccctggggccttcgttcagggcatggtgtaggagcagacagacaaacaccatcccttgcagacaggcactctgagggctattctcttgcaaagataactaagcaccaggccagtaatgggatcctcagactgggcccagaaaaccactctaggaaagttcagggtaggctctctgcaccccctcctcctaatcccgtctccttagtgtctttccgccagcacaggaatgggggaggggtgggtgacgaggatgaacaccggagtccctggaggaagggaagcagggtatctccatctgaggctctgtctttgaggagaggtggagagctggggaagtcttgtgtgaggggattggggctcaggagggggttggggagcaggaagttgtccccaggggagccatcctggcccattcaagggttgagtacttgtttagggttagagctgcccctctggggaccaggattgtccagccaaggccattgtcctgcccccttcccccagtccctcccaggcccctttgaacctgaagtcagatatttcttctctctacccacctcccacccgttgggtttctccacccaggaactaggctggaagctgggatgaggaggtgggggagggagaactgagaatcttgaggaaagaggccccggccttaactgtgaggggatggagcctgggtgcaggtcttatgggggttgggggtggttagtgtctaatctaccaacctgacaacacaagatggatactgtgctctgaaaacgcagagccagcacttctctggggtctctggggacatatctggttggggctcggggtcccatggtgtagagcctctaaactctggaggactggaggtgcaatggctgtcttgtcctggccttggacatgggctgaaatactgggttcacccatatctaggactctagacgggtgggtaagcaagaactgaggagtggccccagaaataattggcacacgaacattcaatggatgttttaggctctccagaggatggctgagtgggctgtaaggacaggccgagagggtgcagtgccaacaggctttgtggtgcgatggggcatccgagcaactggtttgtgaggtgtccggtgacccaaggcaggggtgagaggaccttgaaggttgaaaatgaaggccttcctggggtcccgtcctaagggttgtcctgtccagacgtccccaacctccgtctggaagacacaggcagatagcgctcgcctcagtttctccacccccacagctctgctcctccacccacccagggggcggggccagaggtcaaggctagagggtgggattggggagggagaggtgaaaccgtccc

SEQ ID NO: 9 - DNA, 3734 bp, Homo sapiens
caaatgcgaacttaggctgttacacaactgctggggtctgttctcgccgcccgcccggcagtcaggcagcgtcgccgccgtggtagcagcctcagccgtttctggagtctcgggcccacagtcaccgccgcttacctgcgcctcctcgagcctccggagtccccgtccgcccgcacaggccggttcgccgtctgcgtctcccccacgccgcctcgcctgccgccgcgctcgtccctccgggccgacatgagtggggaccacctccacaacgattcccagatcgaagcggatttccgattgaatgattctcataaacacaaagataaacacaaagatcgagaacaccggcacaaagaacacaagaaggagaaggaccgggaaaagtccaagcatagcaacagtgaacataaagattctgaaaagaaacacaaagagaaggagaagaccaaacacaaagatggaagctcagaaaagcataaagacaaacataaagacagagacaaggaaaaacgaaaagaggaaaaggttcgagcctctggggatgcaaaaataaagaaggagaaggaaaatggcttctctagtccaccacaaattaaagatgaacctgaagatgatggctattttgttcctcctaaagaggatataaagccattaaagagacctcgagatgaggatgatgctgattataaacctaagaaaattaaaacagaagataccaagaaggagaagaaaagaaaactagaagaagaagaggatggtaaattgaaaaaacccaagaataaagataaagataaaaaagttcctgagccagataacaagaaaaagaagccgaagaaagaagaggaacagaagtggaaatggtgggaagaagagcgctatcctgaaggcatcaagtggaaattcctagaacataaaggtccagtatttgccccaccatatgagcctcttccagagaatgtcaagttttattatgatggtaaagtcatgaagctgagccccaaagcagaggaagtagctacgttctttgcaaaaatgctcgaccatgaatatactaccaaggaaatatttaggaaaaatttctttaaagactggagaaaggaaatgactaatgaagagaagaatattatcaccaacctaagcaaatgtgattttacccagatgagccagtatttcaaagcccagacggaagctcggaaacagatgagcaaggaagagaaactgaaaatcaaagaggagaatgaaaaattactgaaagaatatggattctgtattatggataaccacaaagagaggattgctaacttcaagatagagcctcctggacttttccgtggccgcggcaaccaccccaagatgggcatgctgaagagacgaatcatgcccgaggatataatcatcaactgtagcaaagatgccaaggttccttctcctcctccaggacataagtggaaagaagtccggcatgataacaaggttacttggctggtttcctggacagagaacatccaaggttccattaaatacatcatgcttaaccctagttcacgaatcaagggtgagaaggactggcagaaatacgagactgctcggcggctgaaaaaatgtgtggacaagatccggaaccagtatcgagaagactggaagtccaaagagatgaaagtccggcagagagctgtagccctgtacttcatcgacaagcttgctctgagagcaggcaatgaaaaggaggaaggagaaacagcggacactgtgggctgctgctcacttcgtgtggagcacatcaatctacacccagagttggatggtcaggaatatgtggtagagtttgacttcctcgggaaggactccatcagatactataacaaggtccctgttgagaaacgagtttttaagaacctacaactatttatggagaacaagcagcccgaggatgatctttttgatagactcaatactggtattctgaataagcatcttcaggatctcatggagggcttgacagccaaggtattccgtacatacaatgcctccatcacgctacagcagcagctaaaagaactgacagccccggatgagaacatcccagcgaagatcctttcttataaccgtgccaatcgagctgttgcaattctttgtaaccatcagagggcaccaccaaaaacttttgagaagtctatgatgaacttgcaaactaagattgatgccaagaaggaacagctagcagatgcccggagagacctgaaaagtgctaaggctgatgccaaggtcatgaaggatgcaaagacgaagaaggtagtagagtcaaagaagaaggctgttcagagactggaggaacagttgatgaagctggaagttcaagccacagaccgagaggaaaataaacagattgccctgggaacctccaaactcaattatctggaccctaggatcacagtggcttggtgcaagaagtggggtgtcccaattgagaagatttacaacaaaacccagcgggagaagtttgcctgggccattgacatggctgatgaagactatgagttttagccagtctcaagaggcagagttctgtgaagaggaacagtgtggtttgggaaagatggataaactgagcctcacttgccctcgtgcctgggggagagaggcagcaagtcttaacaaaccaacatctttgcgaaaagataaacctggagatattataagggagagctgagccagttgtcctatggacaacttatttaaaaatatttcagatatcaaaattctagctgtatgatttgttttgaattttgtttttattttcaagagggcaagtggatgggaatttgtcagcgttctaccaggcaaattcactgtttcactgaaatgtttggattctcttagctactgtatgcaaagtccgattatattggtgcgtttttacagttagggttttgcaataacttctatattttaatagaaataaattcctaaactcccttccctctctcccatttcaggaatttaaaattaagtagaacaaaaaacccagcgcacctgttagagtcgtcactctctattgtcatggggatcaattttcattaaacttgaagcagtcgtggctttggcagtgttttggttcagacacctgttcacagaaaaagcatgatgggaaaatatttcctgacttgagtgttcctttttaaatgtgaatttttatttctttttaattattttaaaatatttaaacctttttcttgatcttaaagatcgtgtagattggggttggggagggatgaagggcgagtgaatctaaggataatgaaataatcagtgactgaaaccattttcccatcatcctttgttctgagcattcgctgtaccctttaagatatccatctttttctttttaaccctaatctttcacttgaaagattttattgtataaaaagtttcacaggtcaataaacttagaggaaaatgagtatttggtccaaaaaaaggaaaaataatcaagattttagggcttttattttttcttttgtaattgtgtaaaaaatggaaaaaaacataaaaagcagaattttaatgtgaagacattttttgctataatcattagttttagaggcattgttagtttagtgtgtgtgcagagtccatttcccacatctttcctcaagtatcttctatttttatcatgaattcccttttaatcaactgtaggttatttaaaataaattcctacaacttaatggaaa

SEQ ID NO: 10 - DNA, 97 bp, Artificial Sequence
  Description of Artificial Sequence Synthetic oligonucleotide
tgctgttgacagtgagcgcgctgattataaacctaagaaatagtgaagccacagatgtatttcttaggtttataatcagcatgcctactgcctcgga

(19) United States
(12) Patent Application Publication
(10) Pub. No.: US 2011/0244472 A1
(43) Pub. Date: Oct. 6, 2011

(54) Cancer stem cell immortalization

(76) Inventor: Edward A. Prochownik, Pittsburgh, PA (US)

(21) Appl. No.: 12/927,225

(22) Filed: Nov. 10, 2010

Related U.S. Application Data
(63) Continuation-in-part of application No. PCT/US09/43596, filed on May 12, 2009.
(60) Provisional application No. 61/052,846, filed on May 13, 2008.

(51) Int. Cl. C12Q 1/68; C12N 5/095; C12N 5/10; C12N 15/85; C12Q 1/18; G01N 33/574

(52) U.S. Cl. 435/6.13; 435/325; 435/320.1; 435/32; 435/7.23

(57) ABSTRACT

The present invention relates to the preparation and use of immortalized cancer stem cells. The immortalized cancer stem cells of the invention may be used in assays to identify anti-cancer compounds as well as molecules critical to carcinogenesis. Further, cancer stem cells may be harvested from a patient and used, according to the invention, to select agents more likely to be effective in treating the cancer of that particular patient.

### CROSS-REFERENCE TO RELATED APPLICATIONS

This application is a continuation-in-part of International Patent Application No. PCT/US09/43596 filed May 12, 2009 and claims priority to U.S. Provisional Application No. 61/052,846 filed May 13, 2008; the contents of both of these priority applications are hereby incorporated by reference in their entireties herein.

### GRANT INFORMATION

The subject matter of this application was supported, at least in part, by National Cancer Institute Grants RO1CA105033 and RO1CA078259, so that the United States Government has rights herein.

### 1. INTRODUCTION

The present invention relates to the preparation and use of immortalized cancer stem cells. The immortalized cancer stem cells of the invention may be used in assays to identify anti-cancer compounds as well as molecules critical to carcinogenesis. Further, cancer stem cells may be harvested from a patient and used, according to the invention, to select agents more likely to be effective in treating the cancer of that particular patient. Additionally, the class of topoisomerase I inhibitors have been identified as cancer stem cell-specific chemotherapeutic agents.

### 2. BACKGROUND OF THE INVENTION

The concept of cancer stem cells (CSCs) is based upon ideas first formulated in the context of the hierarchical hematopoietic system, where normal stem cells have an unlimited capacity for self-renewal that is gradually lost as they generate multi-potent progenitors and their more differentiated progeny (28). As applied to cancer, the CSC model posits that not all cancer cells are identical in their ability to initiate the growth of a new tumor. In breast cancer, for example, cell surface markers have been used to identify tumor cell subpopulations with widely differing capacities for tumor initiation (1,7). Thus, cells with high surface expression of the epithelial marker CD44 (CD44hi) and low-absent expression of CD24 (CD24lo) possess robust tumor initiating activity relative to the remaining population (1). Recent work has provided evidence that tumors with high levels of CSC markers identify sub-groups of patients who are particularly prone to therapy failure and relapse (20). The concept of CSCs has been extended beyond breast cancer to other malignancies, including cancer of the prostate, brain (gliomas), colon, and pancreas (7, 16-18).

In most cases examined to date, CSCs comprises <1% of tumor cells, with the remainder comprising the more differentiated “transient amplifying cell” (TAC) population. However, the true frequency of CSCs remains somewhat fluid (25). The CSC hypothesis suggests that, because the bulk of tumor cells have low tumor initiating capacity, their eradication will result in substantial tumor shrinkage but will not be curative unless CSCs are eliminated concurrently. This has led to the proposal that relapses arise as a consequence of inherent differential therapeutic sensitivities of the CSC and TAC compartments (15,21,33). Unfortunately, the paucity of CSCs and their tendency to differentiate into TACs has prevented a full-scale testing of this hypothesis. Further, because CSCs differentiate shortly after being isolated, sufficient numbers of CSCs have not been able to be produced via cell culture.

Oct3/4 is a germline-specific transcription factor (50-52, 54-60, also known as OTF3 or POU5F1) recently reported to play an important role in germ cell specification (53). Expression of human Oct3/4 is driven by an approximately 4 kb promoter element (GenBank Acc. No. DQ249177.1; FIG. 9). The Oct3/4 promoter contains three virtual open reading frames (ORFs), all oriented in a 3′→5′ direction relative to the sequence depicted in FIG. 9. ORF1 (ca. 1.1-1.6 kb from the 5′ end) could potentially encode a 162 amino acid protein. ORF2 (ca. 2.4-2.8 kb from the 5′-end) could encode a 138 amino acid protein, and ORF3 (ca. 2.7-3.3 kb from the 5′ end) could encode a 201 amino acid protein. None of these proteins has been previously described nor do they bear significant homology to known GenBank sequences.

### 3. SUMMARY OF THE INVENTION

The present invention relates to immortalized cancer stem cells (“CSCs”). It is based, at least in part, on the discovery that a stably-incorporated fragment of genomic DNA prevents breast CSCs from differentiating, thus allowing for their unlimited expansion, and that these CSCs are significantly more chemo-resistant than transient amplifying cells (“TACs”) to most standard chemotherapeutic agents thus far tested.

In one set of non-limiting embodiments, the present invention provides for cancer stem cells immortalized by the introduction of an Oct3/4 promoter sequence, preferably operably linked to a reporter gene.

In another set of non-limiting embodiments, the present invention provides for assay systems and methods for using such immortalized CSCs to screen for novel small molecules or pre-existing pharmaceuticals that selectively target CSCs.

In another set of non-limiting embodiments, the present invention provides for assay systems and methods for using such immortalized CSCs and siRNAs to identify CSC-specific pathways that are necessary to maintain CSC proliferation and viability.

In yet another set of non-limiting embodiments, the invention may be used to produce an essentially homogeneous population of CSCs.

In yet another set of non-limiting embodiments, the invention may be used to prepare cultures of CSCs from different types of cancers so that the properties of CSCs from these cancers may be compared in terms of phenotype, gene expression profiles, sensitivity to therapeutic agents, and so forth.

In yet another set of non-limiting embodiments, a primary CSC of a subject may be immortalized according to the invention and tested for susceptibility to a cancer therapy agent to evaluate whether said agent would be an effective treatment modality for the subject.

In yet another set of non-limiting embodiments, the present invention provides for assay systems and methods in which CSCs are collected from a subject suffering from a cancer, said CSCs are immortalized and then used to screen potential therapeutic agents so as to select an agent with a greater likelihood of effectiveness against the cancer of that subject.

In yet another set of non-limiting embodiments, various specific compounds, including topoisomerase I inhibitors, may be used to selectively inhibit the growth of CSCs.

### 4. BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A-B. Distinct cloning behaviors of MCF7 tumor cell subsets. (A). MCF7 cells were stained with monoclonal antibodies (mAbs) directed against human CD44 and CD24 (Becton-Dickinson). The anti-CD44 mAb was tagged with Allophycocyanin (APC) and the anti-CD24 mAb was tagged with Phycoerythrin (PE). Depending on the experiment, CSCs (CD44hi/CD24lo) comprised ca. 0.5-1.5% of the entire tumor cell population. (B) Single cell seeding into 96 well plates was accomplished with a DAKO MoFlo Fluorescence-activated cell sorter. Three plates of CD44hi/CD24lo-enriched MCF7 cells and 3 plates of CD44hi/CD24lo-depleted cells were seeded and allowed to grow into macroscopically identifiable colonies. The cumulative number of expandable clones was then noted as soon as they became visible and is plotted as a function of the time after seeding.

FIG. 2. Evaluation of surface marker expression in separate CSC and TAC populations of MCF7 cells. Individual CSC (CD44hi/CD24lo) and non-CSC (CD44hi/CD24lo-depleted or TAC) single cell clones from FIG. 1B were expanded and assessed for the expression of CD44 and CD24. Over 15 clones were examined from each group with 3 representative clones from each group being shown here. Note that clones initially derived from CSCs (CD44hi/D24lo) rapidly reverted so as to now more closely resemble TACs or the uncloned MCF7 cells. The center panel shows the profile of the uncloned, starting MCF7 population, which is similar to that shown in FIG. 1A. Note that in all cases, clones were examined for CD44 and CD24 expression within 3-4 weeks of their initial seeding as single cells.

FIG. 3A-C. Characterization of Oct3/4-GFP+ MCF7-derived CSCs. (A). MCF7 cells were transfected by electroporation with a linearized Oct3/4-GFP vector. A separate cassette on the plasmid imparted G-418 resistance under the control of a promiscuous SV40 promoter. The photos show separate phase contrast and fluorescence micrographs of the same field demonstrating the expected low percentage of GFP+ cells among the stable, G418-resistant transfectants. (B). GFP+ cells from the above population were purified by cell sorting, expanded for several days and examined for CD44 and CD24 expression. Note that these cells were comprised of a highly enriched CSC-like CD44hi/CD24lo population. These cells have remained 100% GFP+ for >20 wk. (C). In the reciprocal experiment, the CD44hi/CD24lo population was isolated from the bulk culture in (A) and then examined for the expression of GFP. Note that virtually all cells expressed GFP (shaded area), a finding confirmed by visual inspection. These cells have all remained GFP+ for >15 wks. As a negative control, non-transfected MCF7 cells were'examined in parallel (open area *).

FIG. 4A-B. Morphological differences between GFP+ CD44hi/CD24lo CSCs and the bulk of MCF7 cells. A. Standard hematoxylin & eosin staining of MCF7 cells and Oct3/4-GFP+MCF7 cells. Cells were plated onto glass coverslips, allowed to attach and grow for two days and then fixed under standard conditions. Light microscopic photos were taken under 60× magnification. Note the relative homogeneity of each population with Oct3/4-GFP+MCF7 cells being smaller and rounder. (B). Size differences between GFP+CD44hi/CD24lo CSCs (dashed curve) and the bulk of MCF7 cells (ribbon curve). Cell diameters for each of the two populations were determined on a Beckman-Coulter Vi-Cell Cell Viability Analyzer. >95% of each population was deemed to be viable and only viable cells were included in the analysis. At least 3000 cells from each group were evaluated Numbers above the curves are the mean diameter of each population

FIG. 5A-C. The Oct3/4-GFP+MCF7 cell population retains its CSC-like properties after in vivo tumorigenesis. (A). Nude mice were inoculated with 10⁴ Oct3/4-GFP+MCF7 cells. 3/3 animals developed tumors within 2 wks. These tumors were allowed to grow for an additional 2 wks at which time they were excised. Single cell suspensions were then prepared and propagated in G-418-containing medium for an additional 2 wk. At the end of this time, the surviving cells were viewed by visible and UV microscopy. (B). The cells were also subjected to flow cytometry to determine the fraction of cells that were positive for GFP. Note that virtually all of the cells retained expression of GFP (shaded curve). A GFP− population of MCF7 cells was included as a negative control (solid curve *). (C). The GFP+ population was also stained for CD44 and CD24. Note that the vast majority of cells displayed the breast cancer CSC phenotype (CD44hi/CD24lo).

FIG. 6. Differential chemotherapeutic responses of MCF7 and Oct3/4-GFP+MCF7 cells. 10⁵ unfractionated, MCF7 cells (dotted line) or Oct3/4GFP+MCF7 cells (broken line) were seeded into 6 well plates and allowed to achieve ca. 30-50% confluence. The indicated concentrations of adriamycin, etoposide, 5-fluorouracil, cis-platinum, methotrexate, and taxol were then added (day 0). The total number of viable adherent cells was then determined at the indicated times by trypan blue exclusion. The results show the average for triplicate plates +/− the SE. Note that in most cases Oct4/4-GFP+MCF7 actually grew in the presence of all agents. These experiments were repeated at least two additional times with similar outcomes.

FIG. 7A-C. Fluorescence-based identification of unfractionated MCF7 cells and Oct3/-GFP+MCF7 CSCs. (A). Unfractionated, non-CSC-MCF7 cells were infected with a lentiviral vector encoding DsRED (Clontech) under the control of the CMV immediate early promoter. They were then mixed with an equivalent number of Oct3/4-GFP+MCF7 cells and photographed with a fluorescence microscope under sequential imaging conditions that captured the individual GFP and DsRED signals. The combined image is shown here. (B). Fluorescence intensities of DsRED+MCF7 cells and Oct3/4-GFP+MCF7 cells are directly proportional to cell number. The indicated numbers of cells were seeded into individual wells of a 96 well plate and allowed to attach overnight. The following day, fluorescence intensities were determined on quadruplicate samples using a fluorescence plate reader (Molecular Devices, SpectraMax M2). Excitation/Emission parameters were 485/538 for GFP and 584/612 for DsRED. The values shown represent the mean fluorescence intensities obtained after background subtraction (<2%). Maximal values, obtained with the highest cell number, were arbitrarily set at 1. (C). DsRED+MCF7 cells and Oct3/4-GFP+MCF7 cells were mixed at the indicated ratios and seeded into 96 well plates. The following day, fluorescence intensities were quantified as described in (B). The black bar indicates the relative fluorescence intensity of the DsRED+ population that was plated in the set of wells labeled 1:1. This number was held constant while the number of GFP+ cells (grey bars) was reduced by the amount indicated.

FIG. 8. Oct3/4 promoter deletion clones that will be used to identify the minimally active region. Schematic diagram of the 3971 bp “full-length” (FL) human Oct 3/4 promoter. The deletions were produced using the corresponding restriction enzyme cleave sites shown in FIG. 9. In particular, the Oct 3/4 promoter sequence of construct B is the NdeI-EcoRI fragment, the Oct 3/4 promoter sequence of construct C is the AccI-BamHI fragment, the Oct 3/4 promoter sequence of construct D is the SacI-BamHI fragment, the Oct 3/4 promoter sequence of construct E is the SalI-BamHI fragment, and the Oct 3/4 promoter sequence of construct F is the NdeI-SalI fragment.

FIG. 9A-B. A 3990-nucleotide human Oct3/4 promoter sequence (GenBank Acc. No. DQ249177.1; SEQ ID NO:1; parts (A) and (B) together contain the entire sequence). The 3′ end of the sequence was operably linked to a nucleic acid encoding GFP in the construct used for MCF7 electroporations. Several pertinent restriction sites that were used for the construction of deletion mutants shown in the accompanying figure are depicted in italics.

FIG. 10. Nucleotide sequence of the murine Oct 3/4 promoter, residues +11 through −1879 from FIG. 2 of Okazawa et al., 1991, EMBO J. 10:2997-3005 (SEQ ID NO:8).

FIG. 11A-C. Selective inhibition of Oct3/4-GFP+ MCF cells with small molecules (A) Rottlerin, (B) A77636 and (C) CGP74514A.

FIG. 12. Structures of (A) Rottlerin, (B) A77636 and (C) CGP74514.

FIG. 13. Schematic diagram showing construction of a lentivirus vector comprising the Neo^{R} gene as well as a ˜4.7 KB Oct3/4-GFP cassette.

FIG. 14A-E. Testing various chemical compounds for CSC-specific inhibitory activity in non-stem MCF7, MDA-MB231 or MDA-MB453 or in Oct3/4-GFP+ CSC cells of those same lines. CSC are denoted by solid circles ()_ and non-stem cells are denoted by solid squares (▪). (A) Testing of A77636, rottlerin, and (β-lapochone. (B) CSC-selective activity of JUN1111 in MDA-MB231 Oct3/4-GFP+ CSC cells. (C) CSC-selective activity of NSC725 in MCF7 Oct3/4-GFP+ CSC cells. (D) CSC-selective activity of NSC743 in MDA-MB453 Oct3/4-GFP+ CSC cells. (E) CSC-selective activity of NSC725 in MDA-MB453 Oct3/4-GFP+ CSC cells.

FIG. 15A-B. Topoisomerase I levels in stem/non-stem cells. (A) Western blot. (B) qRT=PCR.

FIG. 16A-B. Topoisomerase I immunofluorescence (red) in stem and non-stem primary tumor cells. (A) Photomicrograph. (B) Bar graph depiction showing mean fluorescence activity for topoisomerase I in CD49(+) CSC cells and CD49(−) non-stem cells.

FIG. 17. Topoisomerase I expression in topoisomerase I shRNA-treated cells.

FIG. 18. Western blot analysis of topoisomerase I expression in CSC and non-stem cell versions of MCF7, MDA-MB453, BTL12 and MDA-MB231 cells with or without doxycycline-induced (“dox”) shRNA knockdown.

FIG. 19A-C. Nucleic acid sequence of human topoisomerase I (NCBI Ref. Seq. NM_{—}003286.2; SEQ ID NO:9).

FIG. 20A-C. Evaluation of the effect of doxycycline-inducible shRNA targeting topoisomerase I in non-stem MCF7, MDA-MB453 or BTL12 or in Oct3/4-GFP+ CSC cells of those same lines. (A) MCF7. (B) MDA-MB453. (C) BTL12.

FIG. 21A-B. Evaluation of the effect of doxycycline-inducible shRNA targeting topoisomerase I in tumor xenografts generated from (A) non-stem MCF7 cells or (B) Oct3/4-GFP(+) MCF7 CSC cells.

FIG. 22A-E. Effect of various agents on CSC (stem) and non-stem cells from a single primary glioblastoma tumor. (A) CGP-74514A. (B) Rottlerin. (C) A-77636. (D) β-lapachone. (E) Jun1111.

FIG. 23. Western blot showing expression of topoisomerase I in glioblastoma cells and their stem cell countreparts prepared from four different patients.

FIG. 24A-C. Immunofluorescent staining of topoisomerase I (light blue green) and CD133 (red) in primary glioblastoma tumor cells from three individual patients, showing the close correlation between CD133 expression and TopoI staining.

### 5. DETAILED DESCRIPTION OF THE INVENTION

The present invention is based, at least in part, on the discovery that introduction of an Oct3/4 promoter sequence was observed to stabilize the undifferentiated phenotype of cancer stem cells. This effect is alternatively referred to herein as “immortalization”, which, as defined herein, does not require that a culture of such cells would persist indefinitely. Similarly, stability of the undifferentiated phenotype of cancer stem cells, as that phrase is used herein, does not require that all aspects of the cancer stem cell phenotype be retained, but rather that at least one or more characteristics of that phenotype be retained (although not necessarily at the same level as native cells). For example, one or more of the following characteristics are retained: the expression of one or more surface antigen; the level of expression of one or more surface antigen; permeability to a histologic dye; number of cells required to product a tumor when implanted into a host animal; characteristics of tumors produced from the cells; morphology in culture; association with other cells in culture; and sensitivity to pharmacologic agents.

For clarity of description and not by way of limitation, the detailed description of the invention is divided into the following subsections:

(i) Oct 3/4 promoter sequences;

(ii) Oct 3/4 promoter sequence-containing constructs;

(iii) sources of cancer stem cells (“CSCs”);

(iv) assays for therapeutic agents;

(v) assays of interfering RNAs (“iRNAs”);

(vi) CSC drug susceptibility tests;

(vii) reversing the CSC phenotype; and

(viii) selective inhibition of CSC growth.

### 5.1 OCT3/4 Promoter Sequences

“Oct3/4 promoter sequences” are a family of sequences which are related to the native promoter operably linked to a Oct3/4 gene. In one non-limiting embodiment of the invention, the Oct3/4 gene is the human Oct3/4 gene. A variety of lengths of sequences are encompassed within the “Oct3/4 promoter sequences” family. Likewise, some of the sequences exhibit demonstrable promoter activity (for example, of the Oct3/4 gene and/or a reporter gene), whereas others do not. Members of the “Oct3/4 promoter family”, according to the invention, share the property that, when introduced into a cancer stem cell, they promote the persistence of the cancer stem cell phenotype.

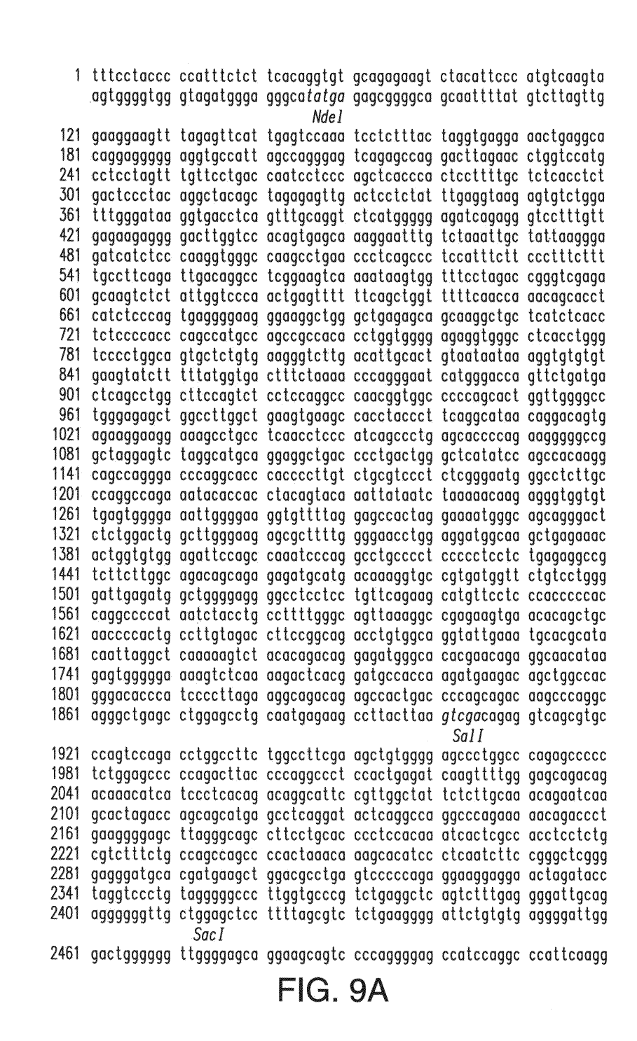

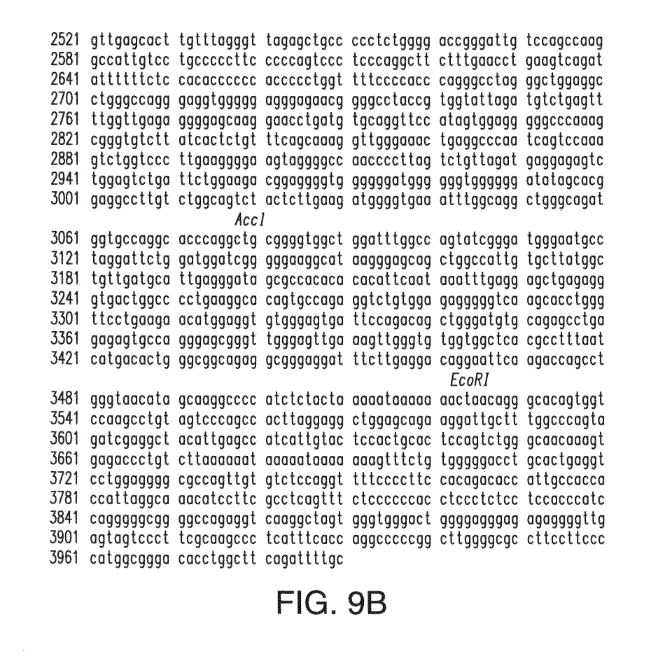

In one specific, non-limiting embodiment, the Oct3/4 promoter sequence is the human nucleic acid sequence set forth in FIG. 9 (GenBank Acc. No. DQ249177) (SEQ ID NO:1), or a sequence which is at least 90 percent, or at least 95 percent, homologous thereto as determined by standard programs, such as BLAST or FASTA

In other non-limiting embodiments, the Oct3/4 promoter sequence does not contain the entire sequence depicted in FIG. 9, but rather a subsequence thereof that, when introduced into a cancer stem cell, promotes the persistence of the cancer stem cell phenotype.

In various non-limiting embodiments, the Oct3/4 promoter sequence is such a subsequence (which is not the entire sequence) which comprises virtual ORF 1 (ca. 1.1-1.6 kb from the 5′ end or a sequence at least 90 percent or at least 95 percent homologous thereto, which could potentially encode a 162 amino acid protein), or virtual ORF2 (ca. 2.4-2.8 kb from the 5′-end or a sequence at least 90 percent or at least 95 percent homologous thereto, which could encode a 138 amino acid protein) or virtual ORF3 (ca. 2.7-3.3 kb from the 5′ end or a sequence at least 90 percent or at least 95 percent homologous thereto, which could encode a 201 amino acid protein), or some combination thereof, such as two of the three virtual ORF. Boundaries of the 3 ORFs in the Oct 3/4 promoter, with translational start and termination sites underlined, are set forth below. These sequences may be comprised in primers for polymerase chain reaction (“PCR”) to amplify the ORF in expressible form.

ORF138 (Starts with CTG)
(SEQ ID NO: 2)
FWD: TGG AAC CTG CAC ATC AGG TTC C
(SEQ ID NO: 3)
REV: TCA GTC TTT GAG GGG ATT GCA
ORF201 (Starts with CTG)
(SEQ ID NO: 4)
FWD: TCC CAG CTG TCT GGA ATC ACT CC
(SEQ ID NO: 5)
REV: CTA CCG TGG TAT TAG ATG TCT G
ORF162 (Starts with ATG)
(SEQ ID NO: 6)
FWD: TAG ATT ATG GGG CCT GGT GG
(SEQ ID NO: 7)
REV: CTA GGA GTC TAG GCA TGC AGG.

In other non-limiting embodiments, the Oct3/4 promoter sequence is a subsequence resulting from cleavage of SEQ NO:1 at the NdeI site shown in FIG. 9 (either the resulting 5′ fragment or the resulting 3′ fragment, or a sequence at least 90 percent or at least 95 percent homologous thereto; or resulting from cleavage of SEQ NO:1 at the SalI site shown in FIG. 9 (either the resulting 5′ fragment or the resulting 3′ fragment, or a sequence at least 90 percent or at least 95 percent homologous thereto; or resulting from cleavage of SEQ NO:1 at the SacI site shown in FIG. 9 (either the resulting 5′ fragment or the resulting 3′ fragment, or a sequence at least 90 percent or at least 95 percent homologous thereto; or resulting from cleavage of SEQ NO:1 at the AccI site shown in FIG. 9 (either the resulting 5′ fragment or the resulting 3′ fragment, or a sequence at least 90 percent or at least 95 percent homologous thereto; or resulting from cleavage of SEQ NO:1 at the EcoRI site shown in FIG. 9 (either the resulting 5′ fragment or the resulting 3′ fragment, or a sequence at least 90 percent or at least 95 percent homologous thereto.

In other non-limiting embodiments, an Oct3/4 promoter sequence is a subsequence of SEQ ID NO:1 consisting essentially of at least 250, or at least 500, or at least 750, or at least 1000, or at least 1250, or at least 1500, or at least 1750, or at least 2000, or at least 2250, or at least 2500, or at least 2750, or at least 3000, or at least 3250, or at least 3500 contiguous residues of SEQ ID NO:1 or a sequence at least 90 percent or at least 95 percent or at least 98 percent homologous thereto.

In further non-limiting embodiments, an Oct3/4 promoter sequence is a subsequence of SEQ ID NO:1, or a sequence at least 90 percent or at least 95 percent or at least 98 percent homologous thereto, which lacks at least 250, or at least 500, or at least 750, or at least 1000, or at least 1250, or at least 1500, or at least 1750, or at least 2000, or at least 2250, or at least 2500, or at least 2750, or at least 3000, or at least 3250, or at least 3500 contiguous residues of SEQ ID NO:1.

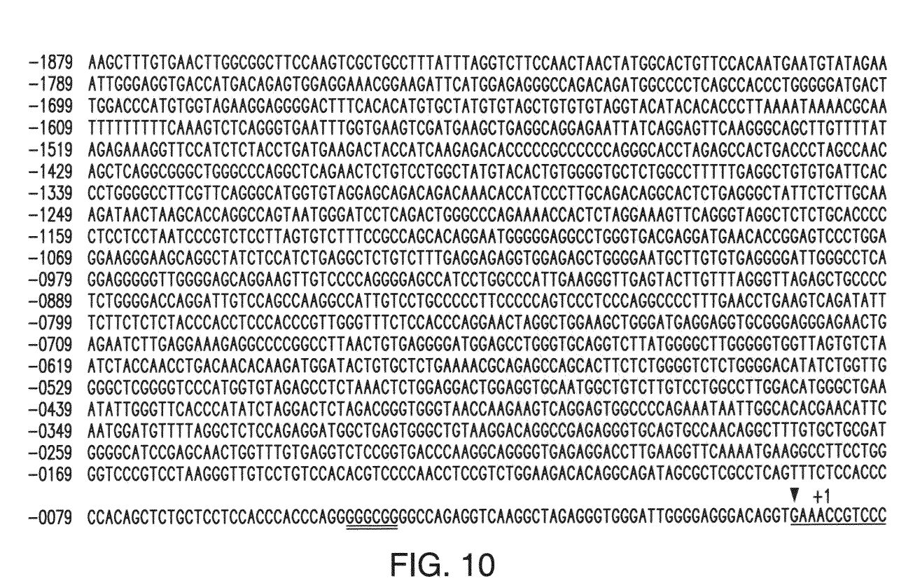

In one specific, non-limiting embodiment, the Oct3/4 promoter sequence is the murine sequence set forth in FIG. 10 (SEQ ID NO:8), or a sequence which is at least 90 percent, or at least 95 percent, homologous thereto as determined by standard programs, such as BLAST or FASTA.

In other non-limiting embodiments, the Oct3/4 promoter sequence does not contain the entire sequence depicted in FIG. 10, but rather a subsequence thereof that, when introduced into a cancer stem cell, promotes the persistence of the cancer stem cell phenotype.

In other non-limiting embodiments, an Oct3/4 promoter sequence is a subsequence of SEQ ID NO:8 consisting essentially of at least 250, or at least 500, or at least 750, or at least 1000, or at least 1250, or at least 1500, or at least 1750, contiguous residues of SEQ ID NO:8 or a sequence at least 90 percent or at least 95 percent or at least 98 percent homologous thereto.

In further non-limiting embodiments, an Oct3/4 promoter sequence is a subsequence of SEQ ID NO:8, or a sequence at least 90 percent or at least 95 percent or at least 98 percent homologous thereto, which lacks at least 250, or at least 500, or at least 750, or at least 1000, or at least 1250, or at least 1500, or at least 1750 contiguous residues of SEQ ID NO:8.

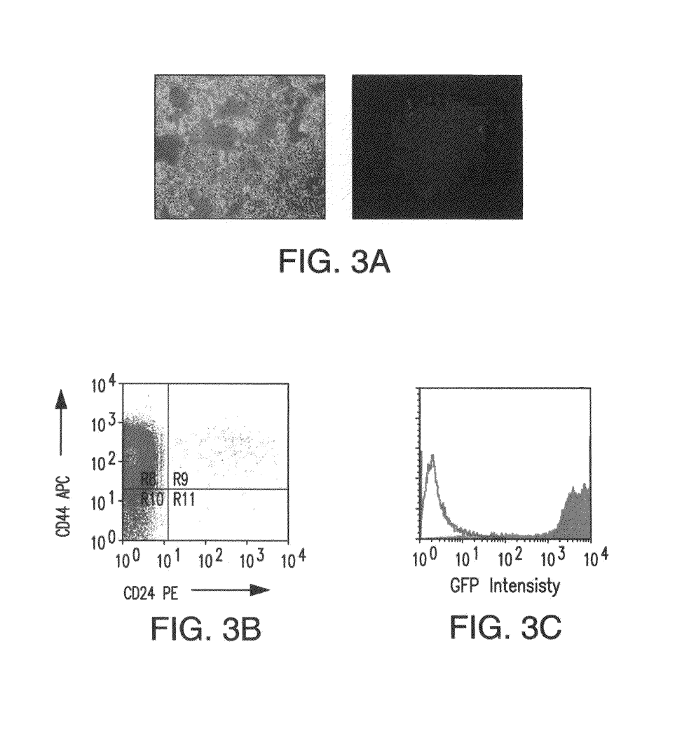

An Oct3/4 promoter sequence for use according to the invention may be identified by using standard techniques to introduce said promoter sequence, optionally operably linked to a reporter gene, into a cancer stem cell, such as a cancer stem cell from a MCF7 cell population, and then determining whether introduction of said sequence results in stabilization of the cancer stem cell phenotype. In a specific, non-limiting example, a putative Oct3/4 promoter sequence may be linearized in a plasmid backbone comprising Neo as a selection marker and electroporated into ca. 5×10⁶ MCF7 cells. The cells may then be selected in G-418 as described in the legend to FIG. 3. Pooled G-418-resistant colonies may then be incubated with mAbs directed against CD44 and CD24 and the CD44hi/CD24lo CSC population may be isolated by cell sorting. The collected cells may then be cultured and the level of stability of the stem cell phenotype may be assessed.

In related embodiments, the present invention provides for an isolated cancer stem cell containing an Oct3/4 promoter sequence which is not operably linked to an Oct 3/4 gene, wherein the cancer stem cell exhibits a stably undifferentiated phenotype. In such embodiments, the Oct3/4 promoter sequence is optionally operably linked to a reporter gene (see the following section).

### 5.2 OCT3/4 Promoter Sequence-Containing Constructs

An Oct3/4 promoter sequence as described in the foregoing section may be comprised in a construct for introduction into a cancer stem cell. In certain, but not all, embodiments, said construct further comprises a reporter gene operably linked to the Oct3/4 promoter sequence.

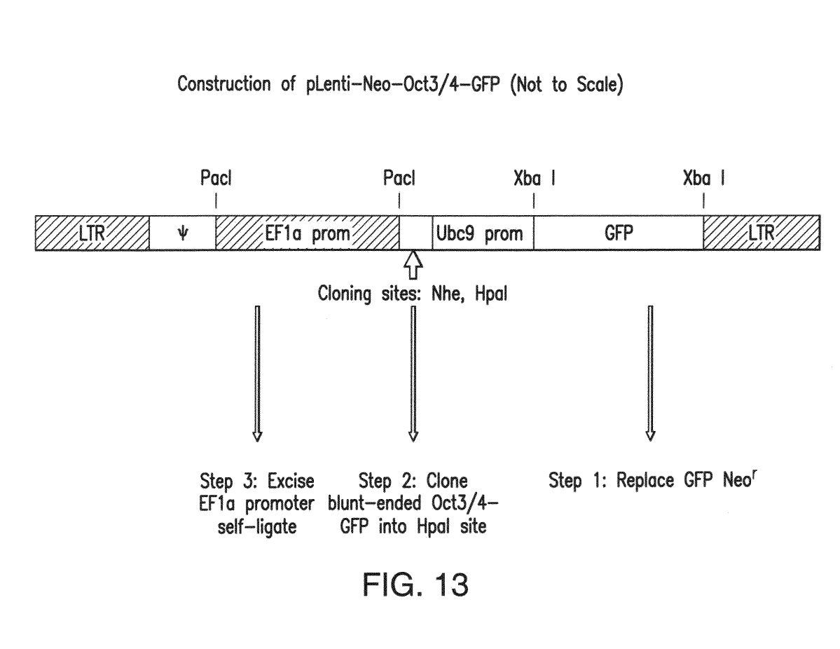

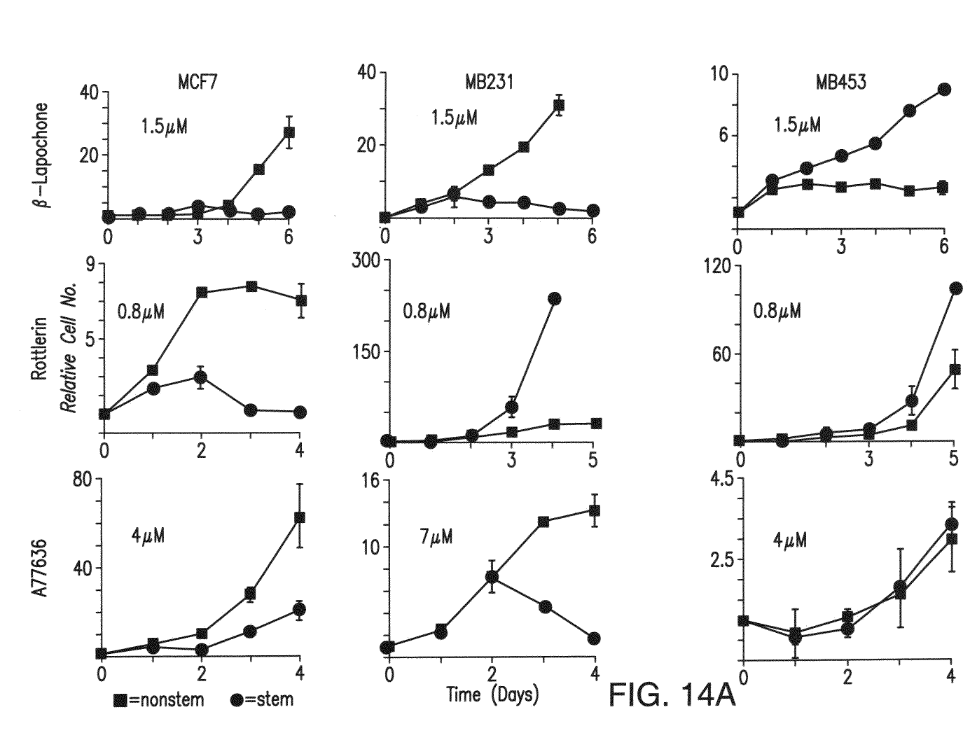

Said construct may be comprised in a vector, such as, but not limited to, a plasmid, a phage, a cosmid, or a virus. Non-limiting examples of viruses which may serve as vectors include lentivirus vectors such as retroviruses, adenovirus, vaccinia virus, adenoassociated virus, etc. Said vector may optionally comprise a gene encoding a selectable product (for example (but not by way of limitation) that confers resistance to an antibiotic) distinct from the reporter gene. Examples of lentivirus vectors are described in Gao et al., 2001, Stem Cells 19:247-259 and Dunlap et al., 2004, J. Invest. Dermatol. 122:549-551, both incorporated by reference herein, wherein the promoter present in such vectors may be replaced by a Oct3/4 promoter-reporter gene construct, e.g. a ˜4.7 kb fragment comprising the Oct3/4 promoter and GFP. A specific non-limiting example of a lentivirus vector is described in section 9, working example 4 and FIG. 13, incorporated by reference into this detailed description section.

Any reporter gene, namely a gene that encodes a detectable product, may be used according to the invention. Preferably the product is detectable in vivo, so that a cancer stem cell containing the construct may be collected based on reporter gene expression and remain viable. Non-limiting examples of suitable reporter genes include a fluorescent protein, such as a Green Fluorescent Protein (“GFP”), enhanced Green Fluorescent Protein (“eGFP”), a Yellow Fluorescent Protein, a Red Fluorescent Protein, etc., a gene that confers antibiotic resistance, or a gene that encodes a cell surface protein not typically found on the cancer stem cell surface, or any gene that encodes a product which confers a detectable phenotype.

In particular non-limiting embodiments, the present invention provides for an isolated nucleic acid comprising an Oct3/4 promoter sequence operably linked to a reporter gene, wherein the Oct3/4 promoter sequence is essentially the entire sequence set forth in FIG. 9 (GenBank Acc. No. DQ249177) (SEQ ID NO:1), or a sequence at least 90 percent, or at least 95 percent, homologous thereto. Said nucleic acid may be contained in a vector.

In other particular non-limiting embodiments, the present invention provides for an isolated nucleic acid comprising an Oct3/4 promoter sequence operably linked to a reporter gene, wherein the Oct3/4 promoter sequence does not contain the entire sequence set forth in FIG. 9 (GenBank Acc. No. DQ249177) (SEQ ID NO:1) (see the preceding section). Said nucleic acid may be comprised in a vector.

In still further non-limiting embodiments, the present invention provides for an isolated nucleic acid which may be used to reversibly immortalize a cancer stem cell, comprising an Oct3/4 promoter sequence operably linked to a reporter gene, wherein said promoter linked to the gene is flanked by lox sites. Said construct may be comprised in a vector. The use of such a construct is discussed in detail below.

In still further non-limiting embodiments, the present invention provides for an isolated nucleic acid comprising an Oct3/4 promoter sequence, and further comprising a gene encoding a selectable marker operably linked to a promoter sequence selectively active in a cancer stem cell (but much less active in a transient amplifying cell), for example a promoter associated with genes such as Cripto, gastrin-releasing peptide receptor, procalyxin-like protein, and hTERT.

In particular, non-limiting embodiments, an Oct3/4 promoter, linked to a reporter gene, may be comprised in a lentivirus vector together with a selectable marker operably linked to a promoter that is active in CSC. A specific, non-limiting embodiment of such a vector is described in section 9, working example 4 and FIG. 13 herein, and includes an Oct3/4 promoter having the sequence set forth in FIG. 9 operably linked to GFP as well as the Neo^{R} gene operably linked to the ubc9 promoter.

### 5.3 Sources of Cancer Stem Cells

A cancer stem cell obtained from any type of cancer may be immortalized—its phenotype stabilized—according to the present invention. The cancer stem cell may be from a human or a non-human subject. The cancer stem cell may be obtained from a tumor cell line or from a primary tumor.

For example, but not by way of limitation, the cancer stem cell may be from a breast cancer or a breast cancer cell line, from a prostate cancer or a prostate cancer cell line, from a glioblastoma or a glioblastoma cell line, from a colon carcinoma or a colon carcinoma cell line, from a lung carcinoma or a lung carcinoma cell line, from a pancreatic cancer or from a pancreatic cancer cell line, from a melanoma or a melanoma cell line, from a gastric cancer or a gastric cancer cell line, from a hepatic carcinoma or a hepatic carcinoma cell line, from an ovarian carcinoma or an ovarian carcinoma cell line, from a testicular cancer or a testicular cancer cell line, from a lymphoma or from a lymphoma cell line, or may be harvested from a leukemia patient or be collected from a leukemia cell line.

A cancer stem cell may be collected by any means known in the art. For example, a cancer stem cell may be collected from (isolated from or enriched from) a larger population of cells using cell surface markers or other properties typical to that cancer stem cell. Alternatively, a Oct3/4 promoter sequence-containing construct which contains a selectable marker which is selectively expressed in a cancer stem cell may be collected from the population via the selectable marker, for example by fluorescence activated cell sorting (“FACS”).

In specific non-limiting embodiments of the invention, expression of the ALDH1 isoform (9a) may be used as a cancer stem cell marker. For example, the Aldefluor Assay (Stem Cell Technologies, Inc.) may be used.

In non-limiting embodiments, where the cancer stem cell is a breast cancer stem cell, a phenotype of cell marker expression CD44hi (meaning increased relative to normal control) and CD24lo (meaning decreased relative to normal control) may be used to collect cancer stem cells (for example, using antibodies directed to said proteins and FACS). Where a cell line is used as the source of cancer stem cells, suitable cell lines include, but are not limited to, MCF7, T-47D, UACC-812, HCC38, HCC1428, SKBR-3, MB-157, BTL 12, MDA-MB231 and MDA MB453.

In non-limiting embodiments, where the cancer stem cell is a colon cancer stem cell, a phenotype of cell marker expression EpCAMhi/CD44hi, or expression of CD133, or the ability to exclude the dye Hoechst 33342, may be used to collect cancer stem cells (12, 27, 31, 38). Where a cell line is used as the source of cancer stem cells, suitable cell lines include, but are not limited to Colo320, HCT15, and SW480.

In non-limiting embodiments, where the cancer stem cell is a prostate cancer stem cell, a phenotype of cell marker expression CD44hiCD24lo/ScaI+ or the ability to exclude the dye Hoechst 22243, may be used to collect cancer stem cells (5,7,44). Where a cell line is used as the source of cancer stem cells, suitable cell lines include, but are not limited to PC3, DU145, and LNCaP.

In non-limiting embodiments, where the cancer stem cell is a pancreatic cancer stem cell, a phenotype of cell marker expression CD44hi, CD24hi, ESAhi (18) may be used to collect cancer stem cells. Where a cell line is used as the source of cancer stem cells, suitable cell lines include, but are not limited to PANC-1 and ASPC-1.

### 5.4 Assays for Therapeutic Agents

The present invention provides for assays in which the immortalized cancer stem cells may be used to identify useful therapeutic agents, by screening various test agents. The test agents may be known bioactive compounds or may be compounds without hitherto known biological activity. Suitable test agents may also be biologic molecules, including but not limited to proteins, antibodies or antibody fragments, oligonucleotides, peptidomimetic compounds, etc.

In non-limiting embodiments, the present invention provides for a method of identifying an anti-cancer agent, comprising:

(i) providing an isolated cancer stem cell containing an Oct3/4 promoter sequence which is not operably linked to an Oct3/4 gene;

(ii) providing a means for evaluating the proliferation, differentiation level, and/or viability of the cancer stem cell;

(iii) administering a test agent to the cancer stem cell; and

(iv) evaluating the proliferation and/or differentiation and/or viability of the cancer stem cell;

wherein an inhibition of proliferation, increase in level of differentiation, or decrease in viability associated with the presence of the test agent indicates that the test agent is an anti-cancer agent. In certain non-limiting embodiments of this method, the means for evaluating the proliferation, differentiation level, and/or viability comprises measuring and/or detecting expression of a reporter gene (see above).

In certain non-limiting embodiments, the present invention provides for a method of identifying an anti-cancer agent with selective activity toward cancer stem cells, comprising:

(i) providing a population of cancer cells comprising cancer stem cells as well as cancer cells which are not stem cells, where the relative proportions of cancer stem cells and cancer cells which are not stem cells is known;

(ii) administering a test agent to the population of cells;

(iii) culturing the population after (ii); and

(iv) determining the relative proportions of cancer stem cells and cancer cells which are not stem cells in the population after (iii);

wherein a decrease in the relative proportion of cancer stem cells indicates that the test agent is an anti-cancer agent with selective activity against cancer stem cells. In non-limiting embodiments of this method, there is a first means for detecting a cancer stem cell and a second, different means for detecting a cancer cell that is not a stem cell, where said first means and said second means are used to determine the relative proportions of cancer stem cells and cancer cells which are not stem cells. For example, in non-limiting embodiments said first means and/or second means may be a fluorescent antibody to a cell surface antigen (if both means are fluorescent antibodies, they are desirably of different colors). Alternatively, in other non-limiting embodiments, said first means and/or second means may be an expression construct having a reporter gene selectively expressed in a cancer stem cell or a cancer cell which is not a stem cell (if both means are reporter genes, they preferably encode different products). As a specific non-limiting example, a cancer stem cell may be detected via a detectable reporter gene (e.g. a fluorescent protein of a first color) operably linked to a Oct3/4 promoter sequence with promoter activity, and a cancer cell which is not a stem cell may be detected by a fluorescent antibody (of a second color) which recognizes a surface antigen present on said cancer cell but absent or in substantially lower amounts on a cancer stem cell.

In non-limiting embodiments, high throughput screening techniques may be used.

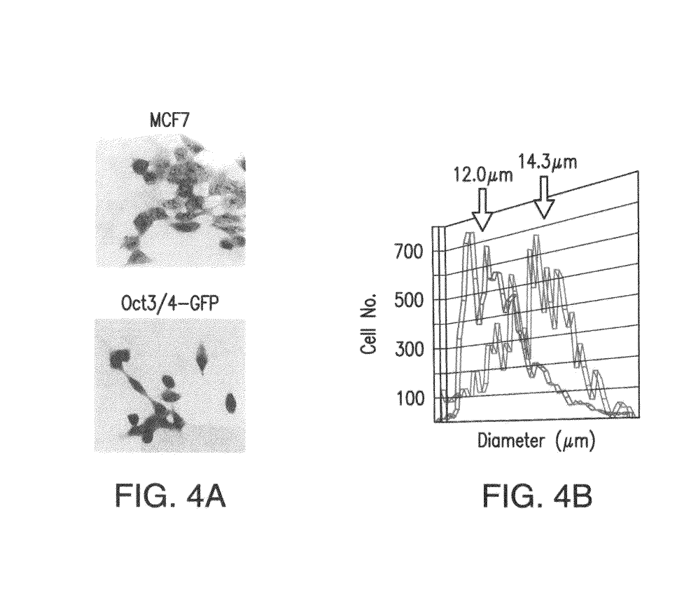

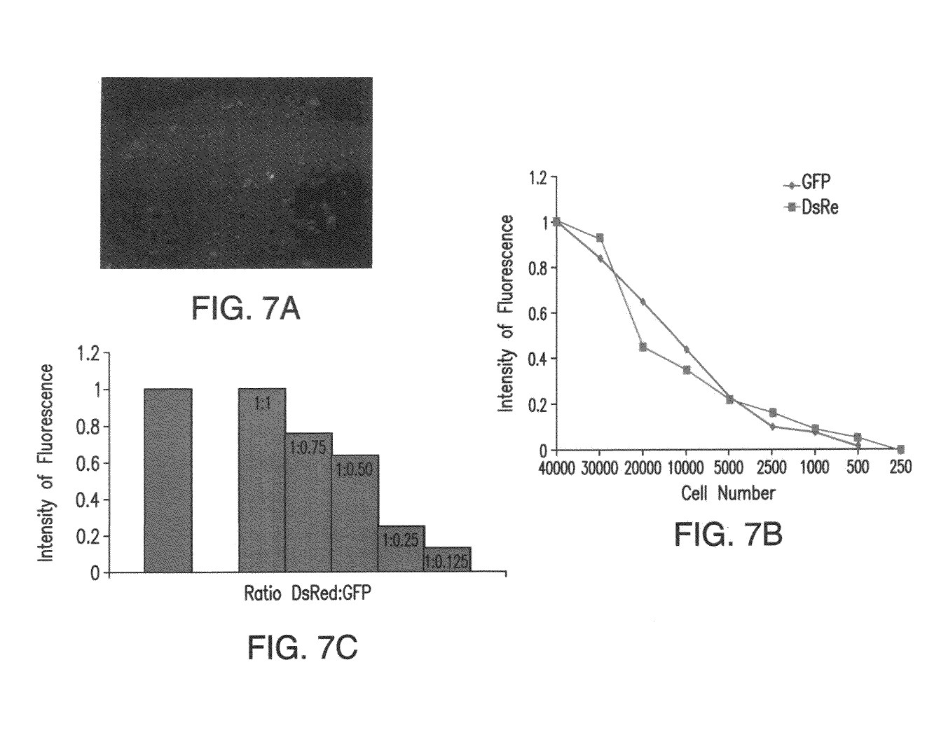

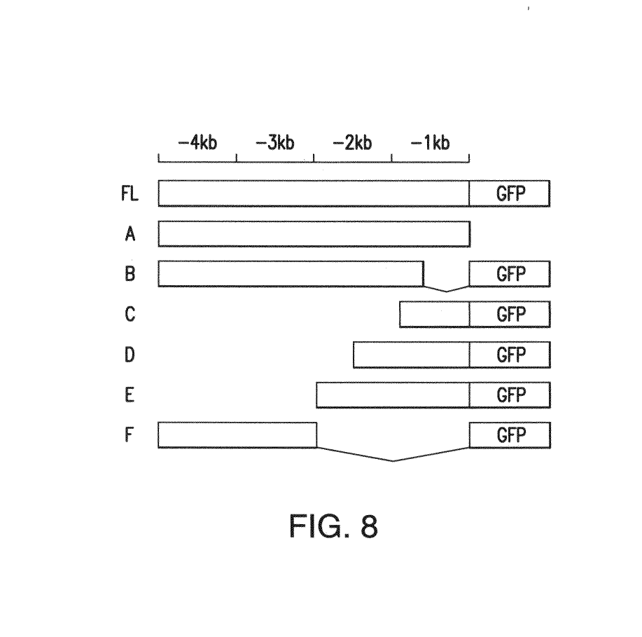

In a specific, non-limiting example, the present invention provides for an assay for screening low molecular weight compounds (“LMWCs”, having a molecular weight of less than about, for example and not by way of limitation, 600 daltons), as follows. Unfractionated, non-CSC-MCF7 cells may be infected with a lentiviral vector encoding DsRED (Clontech). CSC-MCF7 cells may separately be transfected or infected with an expression construct comprising an Oct3/4 promoter operably linked to GFP. In the assay, LMWC may be identified which inhibit proliferation/survival of GFP+CSCs relative to control DsRed-tagged MCF7 cells. The screening procedure may use a 1:1 mix of the above-described GFP and DsRed cells. After robotically dispensing a total of ˜2000 cells into 384 well plates, they may be incubated overnight to allow attachment. LMWCs (each in DMSO) may then be added to each well to a final compound concentration of 10 μM (final DMSO concentration <1% which is easily tolerated). GFP:DsRed ratios may then be determined daily over the ensuing 2-3 days (see FIG. 7). All compounds that reduce GFP:DsRED ratios >3SDs below the mean of the control (No LMWC added; DMSO vehicle only) may be flagged for subsequent follow up. Further refinement of the “hits” may utilize advanced mathematical methods such as B-scores and BZ-scores to reduce the false positive rate even lower (48,49). The foregoing assay, while sensitive, may lack specificity as there are several ways that a compound could alter the GFP:DsRED ratio other than by inhibiting the proliferation or survival of the GFP+ population: for example, (i) it could inhibit or quench the fluorescence intensity of GFP (specific or non-specific but not of interest); (ii) it could promote the growth of the DsRED population (specific but likely not of interest); (iii) it could enhance the fluorescence of DsRED (specific or non-specific but not of interest); or (iv) it could promote the differentiation of the GFP+ population into TACs associated with concurrent down-regulation of the Oct3/4 promoter (specific and of interest). Most of these possibilities would not be expected to be distinguishable on the initial screen. Therefore, a compounds flagged in this “first pass” assay would preferably be subjected to further testing, for example re-testing in triplicate. Repeat hits may then be examined at serial dilutions to establish ID50's and to identify compounds that are active at submicromolar concentrations. The lowest concentration of compounds that maximally inhibit the GFP signal may then be re-screened with isolated populations of Oct3/4-GFP+MCF7, DsRED-MCF7 cells and an additional line of MCF7 cells that expresses GFP under the control of a neutral (CMV) promoter. Growth curves for each population may be determined, and apoptosis assays (TUNEL assays, Annexin V staining, and/or caspase-3 cleavage [Caspase3/7, Promega]) may be performed. Visual inspection of cells, flow cytometry and cell sizing using a Vi-Cell apparatus (FIG. 4) may be used to determine whether loss of GFP expression in individual cells is occurring as would be expected if the compound were promoting CSC differentiation that might, without affecting cell proliferation or viability, cause loss of GFP due to a differentiation-mediated down-regulation of the Oct3/4 promoter. H&E and CD44/CD24 staining may be used to confirm this by documenting changes in morphology and cell surface phenotype.

Section 8 herein describes a working example of an assay which tested the growth inhibitory effects of various small molecules on breast cancer CSC having an Oct3/4 promoter-GFP construct introduced via a lentivirus vector. As per that examples, an assay may be conducted using the Promega CellTiter-Blue® Cell Viability Assay (Promega, Madison, Wis. As one non-limiting example, cells may be distributed into a 384-well plate so that 30 ul medium is in each well, to which 6 ul of CellTiter-Blue may be added. The plate may then be, incubated at 37° C. for 2.5 hours, and then read on a SpectraMax M5 Microplate Reader using the wavelength of excitation 560 nm/emission 590 nm.

### 5.5 Assays of Interfering RNAs

In non-limiting embodiments, the present invention provides for a method of identifying a gene associated with the cancer stem cell phenotype, comprising:

(i) providing an isolated cancer stem cell containing an Oct3/4 promoter sequence which is not operably linked to an Oct3/4 gene;

(ii) providing a means for evaluating the proliferation, differentiation level, and/or viability of the cancer stem cell;

(iii) administering a test interfering RNA to the cancer stem cell; and

(iv) evaluating the proliferation and/or differentiation and/or viability of the cancer stem cell;

wherein an inhibition of proliferation, increase in level of differentiation, or decrease in viability associated with the presence of the interfering RNA indicates that the test agent is an anti-cancer agent. According to such methods, the means for evaluating the proliferation, differentiation level, and/or viability may comprise measuring and/or detecting expression of a reporter gene.

siRNA (or shRNA)-mediated transcript “knockdown,” a technique which has emerged as a standard way of specifically and potently inhibiting the expression of large numbers of genes (3,9,23,34), may be used in the foregoing method. This efficient and simple approach allows the functions of protein encoded by the targeted transcripts to be inferred from the observed phenotypic changes of the recipient cells. Moreover, unlike drug screening approaches, the target is immediately knowable. Genome-wide siRNA/shRNA screens have allowed large-scale and/or multiplex targeting that potentially allows for the identification of all genes required for a particular phenotype (35,37). For example, one recent screen has uncovered >250 cellular factors involved in HIV infection (4), with many not having been previously recognized as being relevant to viral pathogenesis. An additional advantage of siRNA/shRNA-based screens is that they can implicate relevant transcripts whose levels do not change between two cells and thus would not be identifiable by methods such as transcriptional profiling. Knowing that a particular protein was required for the maintenance of proliferation/viability of a CSC might indicate that the same protein, or others in its pathway, are also targeted by

LMWCs. This could provide a starting point to enable the otherwise unknown mechanism of action of these compounds to be determined.

In a specific, non-limiting embodiment of the invention, the foregoing method may be applied using the Ambion Version 2.0 siRNA library (Austin, Tex.), which comprises 16,560 unique siRNAs targeting 5,520 distinct “drug-able” human transcripts (not included for example are siRNAs targeting mRNAs for many structural proteins). To reduce cost and effort, the three siRNAs for each transcript may be combined in a single well for the initial screen. “Hits” may be re-screened with each of the three individual siRNAs. This two-step process maximizes the likelihood that each targeted transcript will be effectively silenced while minimizing the size and cost of the initial screen. siRNAs may be added to cells at a final concentration of 5-10 nM using either siPORT Amine or siPORT Lipid Transfection Agents (Ambion). GFP:DsRED ratios may be determined daily (e.g., for 3 days). To this end, admixed cultures Oct3/4-GFP+MCF7 cells and DsRED+MCF7 cells may be re-screened with candidate siRNAs identified in the initial survey. Those which reproduce the reduction in GFP:DsRED ratio may be tested in triplicate again with individual cultures of Oct3/4-GFP+MCF7 CSCs and DsRED-MCF7s, and proliferation, apoptosis, and differentiation may be evaluated. As another confirmatory experiment, stable expression of short hairpin RNA (shRNAs) in the targets cells may be used.

As a further confirmatory test. an siRNA-resistant form of the targeted transcript may be expressed in Oct3/4-GFP+MCF7 cells, and it may be determined whether these cells can be rescued from the effects of the siRNA. For example, a bicistronic lentiviral or retroviral vector may be used that expresses the coding sequence of the targeted human transcript (32). Two silent nucleotide changes may be created in adjacent 3rd position codons of the sequence targeted by the siRNA. The resultant mismatches between the siRNA and its target would be expected to be sufficient to allow the exogenous transcript to escape inhibition without altering its encoded protein sequence. The vector may be transduced into Oct3/4-GFP+MCF7 cells, which may then be selected in an appropriate antibiotic (blasticidin, hygromycin, or puromycin since the cells are already G418-resistant).

Proteins encoded by the targeted mRNAs may be compared using protein function-interaction software such as LocusLink (www.ncbi.nlm-nih.gov/LocusLink), Pathfinder (2) and PathwayArchitect (Stratagene) in order to determine the number and nature of the pathways being inhibited and their inter-relationships.

### 5.6 Cancer Stem Cell Drug Susceptibility Tests

In non-limiting embodiments, the present invention provides for a means of identifying an agent likely to be of benefit to a subject, where the subject has a cancer, comprising:

(i) collecting a cancer stem cell from the subject;

(ii) introducing, into the cancer stem cell from the subject, a nucleic acid comprising as Oct3/4 promoter sequence operably linked to a reporter gene;

(iii) exposing the product of step (ii) to an agent; and

(iv) determining whether exposure to the agent inhibits proliferation, increases the level of differentiation, and/or decreases the viability of the product of step (ii),

where an inhibition of proliferation, increase in the level of differentiation, or decrease of viability indicates that the agent may be of therapeutic benefit to the subject. This method may be desirably practiced for a number of different agents, and selecting the agent which most effectively, among those tested, inhibits proliferation, increases the level of differentiation, and/or decreases viability of the cancer stem cell containing the nucleic acid comprising an Oct3/4 promoter sequence operably linked to a reporter gene. See, for example, section 8, working example 3 below.

### 5.7 Reversing the Cancer Stem Cell Phenotype

In non-limiting embodiments, the present invention provides for a vector which provides the means for removing the Oct3/4 promoter sequence, so that the immortalized phenotype may be reversed. In a specific, non-limiting example, a vector in which the full-length Oct3/4-GFP sequence is flanked by LoxP sites (“foxed”). This vector may be electroporated into MCF7 cells and G-418-resistant stable clones may be selected and pooled. The GFP+ cells may be purified by cell sorting, expanded, and cell surface phenotyped. The expanded GFP+ CSC population may next be transfected with expression vectors for Cre recombinase.

In such an expression vector, Cre may be operably linked to a suitable promoter element. The promoter element may be constitutively active, or selectively active under certain conditions. In non-limiting embodiments, the promoter may be an inducible promoter, for example the murine mammary tumor virus promoter (inducible with dexamethasone); commercially available tetracycline-responsive or ecdysone-inducible promoters, etc. (Romano, Drug News Perspect (2004) 17(2):85-90). In specific non-limiting embodiments of the invention, the promoter may be selectively active in cancer cells, such as the prostate specific antigen gene promoter (O'Keefe et al. (2000) Prostate 45:149-157), the kallikrein 2 gene promoter (Xie et al. (2001) Human Gene Ther 12:549-561), the human alpha-fetoprotein gene promoter (Ido et al. (1995) Cancer Res 55:3105-3109), the c-erbB-2 gene promoter (Takakuwa et al. (1997) Jpn. J. Cancer Res. 88:166-175), the human carcinoembryonic antigen gene promoter (Lan et al. (1996) Gastroenterol. 111:1241-1251), the gastrin-releasing peptide gene promoter (Inase et al. (2000) Int. J. Cancer 85:716-719). the human telomerase reverse transcriptase gene promoter (Pan and Koenman, 1999, Med Hypotheses 53:130-135), the hexokinase II gene promoter (Katabi et al. (1999) Human Gene Ther 10:155-164), the L-plastin gene promoter (Peng et al. (2001) Cancer Res 61:4405-4413), the neuron-specific enolase gene promoter (Tanaka et al. (2001) Anticancer Res 21:291-294), the midkine gene promoter (Adachi et al. (2000) Cancer Res 60:4305-4310), the human mucin gene MUC1 promoter (Stackhouse et al. (1999) Cancer Gene Ther 6:209-219), and the human mucin gene MUC4 promoter (Genbank Accession No. AF241535), which is particularly active in pancreatic cancer cells (Perrais et al. (2000) J Biol Chem 276(33):30923-30933).

In a specific, non-limiting embodiment, Cre may be driven by a CMV immediate-early viral promoter or a PGK promoter (both obtained from Dr. Richard Chaillet, The University of Pittsburgh Dept. of Microbiology and Molecular Genetics). The advantage of the first vector is its potentially high level expression. A theoretical disadvantage is that it may not be well-expressed in CSCs since the CMV promoter is reported to function poorly in embryonal stem cells. In contrast, the PGK promoter may drive lower levels of Cre recombinanse but is routinely used to express various genes in embryonal stem cells. Following transfection, cells may be monitored for the expression of GFP. Once the GFP-negative population is observed, however, it may be isolated by standard cell sorting. As a control, the GFP+ population from the same culture, indicative of cells that have not been transfected with the Cre expression vector, may be separately isolated. Following the expansion of both populations, they may be evaluated for the expression of CD44, CD24, and ALDH1; for stem cell-specific transcripts by qRT-PCR; for Hoechst 33342 “side populations”; for in vivo tumor initiating activity; and for sensitivity to chemotherapeutic drugs.

### 5.8 Selective Inhibition of CSC Growth

In non-limiting embodiments, the present invention provides for methods of selectively inhibiting the growth of a CSC, comprising exposing the CSC to an effective amount of a compound that selectively inhibits CSC cell growth (meaning that the effect of the compound on growth of the CSC cell (measured as proliferation and/or viability) is proportionally greater than the effect of the compound on growth of a non-stem cell equivalent of the CSC. For example, but not by way of limitation, the compound may be CGP-74514A (which may be, for example, at a concentration of at least about 0.1 μM or at least about 0.5 μM or at least about 1 μM), or rottlerin (which may be, for example, at a concentration of at least about 0.1 μM or at least about 0.5 μM or at least about 1 μM), or A-77636 (which may be, for example, at a concentration of at least about 0.1 μM or at least about 0.5 μM or at least about 1 μM) or β-lapachone (which may be, for example, at a concentration of at least about 0.1 μM or at least about 0.5 μM or at least about 1 μM) or Jun1111 (which may be, for example, at a concentration of at least about 0.1 μM or at least about 0.5 μM or at least about 1 μM).

In certain embodiments, the present invention provides for a method of selectively inhibiting the growth of CSCs in a population of cancer cells, comprising exposing the population to an effective amount of a compound that selectively inhibits CSC growth.

In certain embodiments, the present invention provides for a method of treating a subject suffering from a cancer, where the cancer contains CSCs, comprising administering, to the subject, an effective amount of a compound that selectively inhibits CSC growth.

In certain embodiments, the present invention provides for a method of selectively inhibiting the growth of a cancer stem cell, comprising exposing the cancer stem cell to a compound selected from the group consisting of NSC725, NSC743 and JUN111 in an amount that inhibits growth of the cancer stem cell.

In certain embodiments, the present invention provides for a method of selectively inhibiting the growth of cancer stem cells in a population of cancer cells, comprising exposing the population to a compound selected from the group consisting of NSC725, NSC743 and JUN1111 in an amount that inhibits the growth of the population of cancer cells.

In certain non-limiting embodiments, the present invention provides for a method of treating a subject suffering from a cancer comprising administering, to the subject, a compound selected from the group consisting of NSC725, NSC743 and JUN1111 in an amount that inhibits the growth of the cancer.

In non-limiting embodiments, the present invention provides for methods of selectively inhibiting the growth of a CSC, comprising exposing the CSC to an effective amount of a topoisomerase I inhibitor.

In certain embodiments, the present invention provides for a method of selectively inhibiting the growth of CSCs in a population of cancer cells, comprising exposing the population to an effective amount of a topoisomerase inhibitor.

In certain embodiments, the present invention provides for a method of treating a subject suffering from a cancer, where the cancer contains CSCs, comprising administering, to the subject, an effective amount of a topoisomerase I inhibitor.

Non-limiting examples of topoisomerase I inhibitors include β-lapochone, NSC725 and NSC743 (see Antony S, Agama K K, Miao Z H, Takagi K, Wright M H, Robles A I, Varticovski L, Nagarajan M, Morrell A, Cushman M, Pommier Y. Cancer Res. 2007 Nov. 1; 67(21):10397-405; and Holleran J L, Parise R A, Yellow-Duke A E, Egorin M J, Eiseman J L, Covey J M, Beumer J H., J Pharm Biomed Anal. 2010 Sep. 5; 52(5):714-20).

Additional non-limiting examples of topoisomerase I inhibitors include: camptothecin and irinotecan.

In a specific, non-limiting embodiment, β-lapochone may be administered to achieve a concentration local to the target CSC of at least about 1.5 μM.

In a specific, non-limiting embodiment, NSC725 may be administered to achieve a concentration local to the target CSC of at least about 1 nM or at least about 5 nM or at least about 7 nM or at least about 10 nM.

In a specific, non-limiting embodiment, NSC743 may be administered to achieve a concentration local to the target CSC of at least about 1 nM or at least about 5 nM or at least about 10 nM

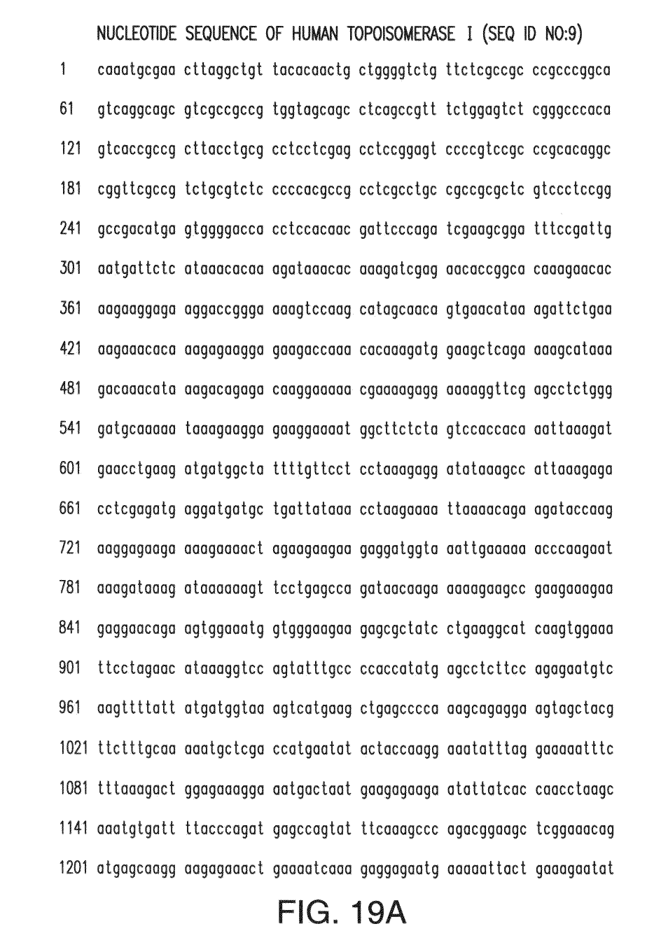

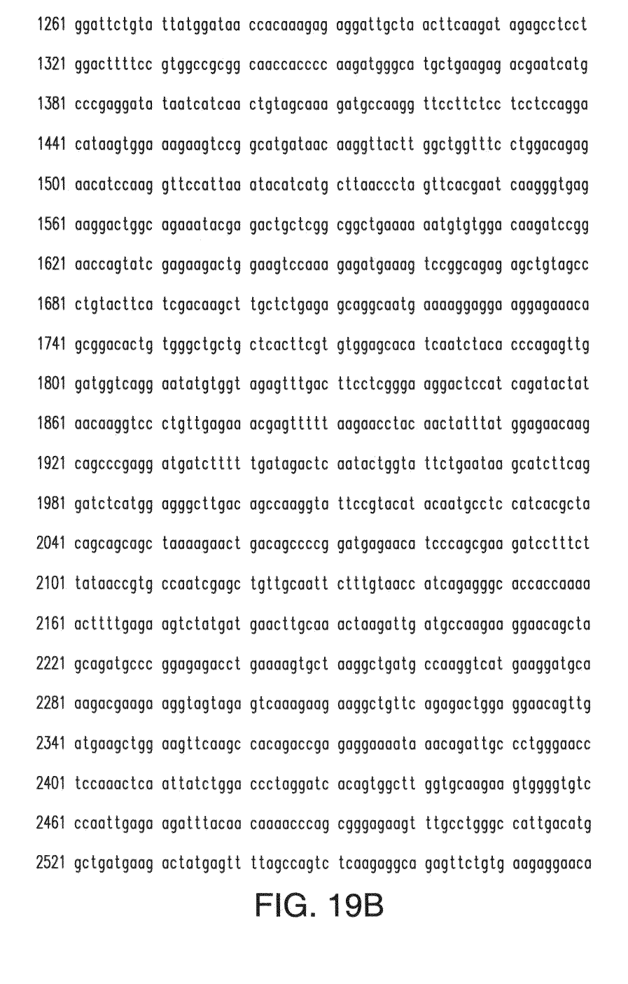

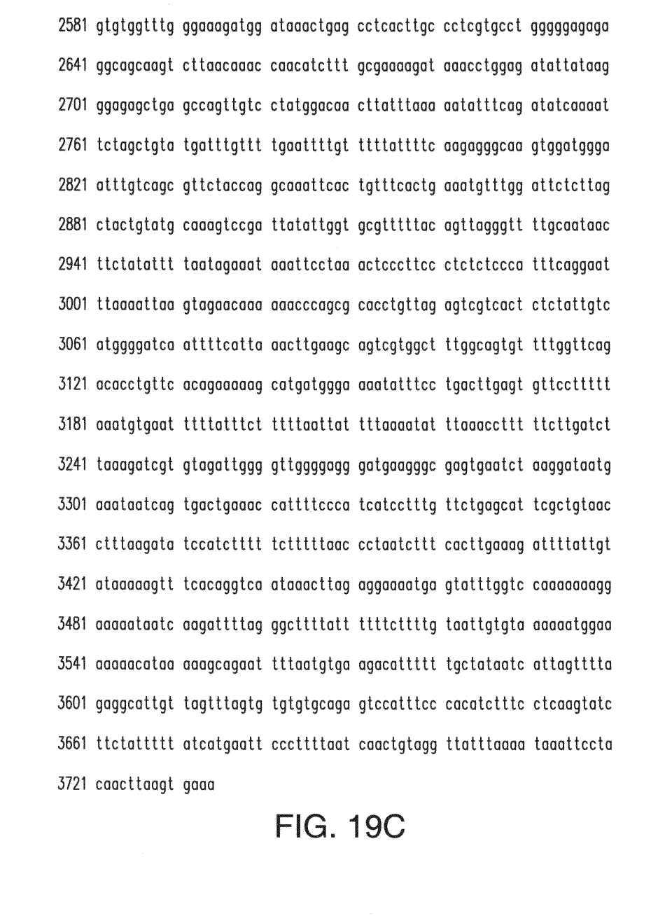

In further non-limiting embodiments, antisense or siRNA directed against the topoisomerase I gene may be used as the topoisomerase I inhibitor (for general references see WO01/75164 or U.S. Pat. No. 7,078,196). For example, antisense or siRNA may be produced directed against the human topoisomerase I gene or a topoisomerase I gene of a non-human subject in which topoisomerase I is desirably inhibited), where the sequence of human topoisomerase I is set forth in SEQ ID NO: 9 (FIG. 19). As a specific, non-limiting example, a siRNA or shRNA may comprise at least about 15, or at least about 20, or at least about 25, nucleotides of:

(SEQ ID NO: 10)
TGCTGTTGACAGTGAGCGCGCTGATTATAAACCTAAGAAATAGTGAAGCC
ACAGATGTATTTCTTAGGTTTATAATCAGCATGCCTACTGCCTCGGA.

In certain embodiments of the invention the antisense or siRNA may be between about 5 and 50 or between about 15 and 40 or between about 15 and 30 nucleotides in length.

Any of the foregoing compounds may be administered by any route known in the art, including oral, intravenous, intraperitoneal, intrathecal, intrarterial, intravesicle, nasal, by pulmonary inhalation, or by direct injection/instillation into a tumor site.

The above methods may be used to treat a cancer and/or cancer stem cells that are breast cancer, glioblastoma, prostate cancer, ovarian cancer, esophageal cancer, pancreatic cancer, gastric cancer, colon cancer, lung cancer, liver cancer, melanoma, leukemia, or lymphoma. In specific non-limiting embodiments, the present invention may be used to treat a cancer where CSCs of the cancer exhibit an elevation in the level of topoisomerase I, for example relative to non-stem cells of the cancer.

### 6. WORKING EXAMPLE 1

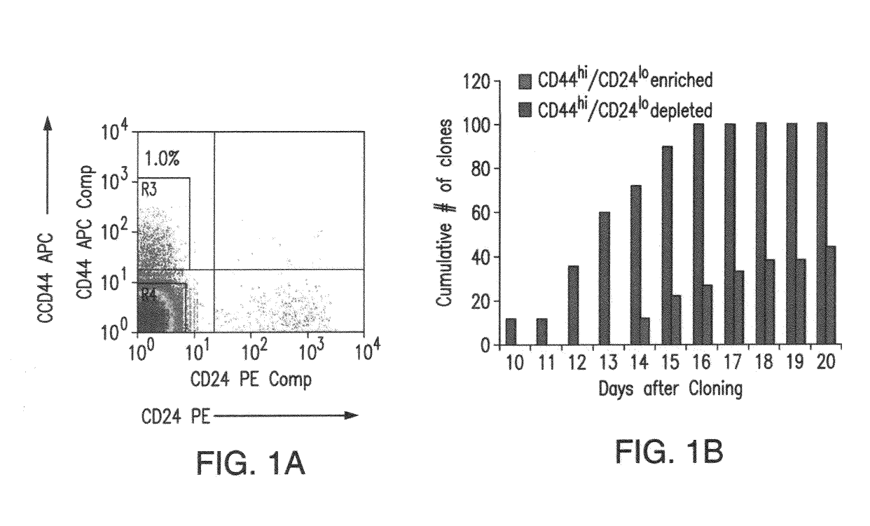

1. Breast cancer stem cells have a lower cloning efficiency and arise more slowly than TACs. MCF7 breast cancer cells were assessed for CD44 and CD24 expression, which can be used to identify and isolate CSCs (1). As shown in FIG. 1A, CD44hi/CD24lo cells comprised ca. 1% of the entire population, in keeping with previous observations in both MCF7 cells and primary cancers (1,46). These cells were then sorted into 96 well plates and expanded as single cell clones. The remainder of the population, consisting of the bulk of the cells, and comprised almost exclusively of TACs, was similarly sorted into a separate set of 96 well plates. All plates were examined daily and the point at which macroscopically visible colonies first became detectable was noted. As seen in FIG. 1B, the TAC population showed a cloning efficiency of 34%; clones were first detected 10 days after plating, and no additional clones were detected beyond day 16. In contrast, CSCs cloned less efficiently (14%), were not first detected until day 14, and continued to appear until day 20. Very similar results were obtained in two repeat experiments. These findings are consistent with a previous report that MCF7 CSCs express higher levels of genes associated with quiescence (46). It also suggests that, at least when isolated as single cells, CSCs may initially divide more slowly than TACs, as evidenced by the delayed timing of their clonal expansion. Once reaching this multi-cell stage, however, no significant differences were observed in the growth rates of these two populations.

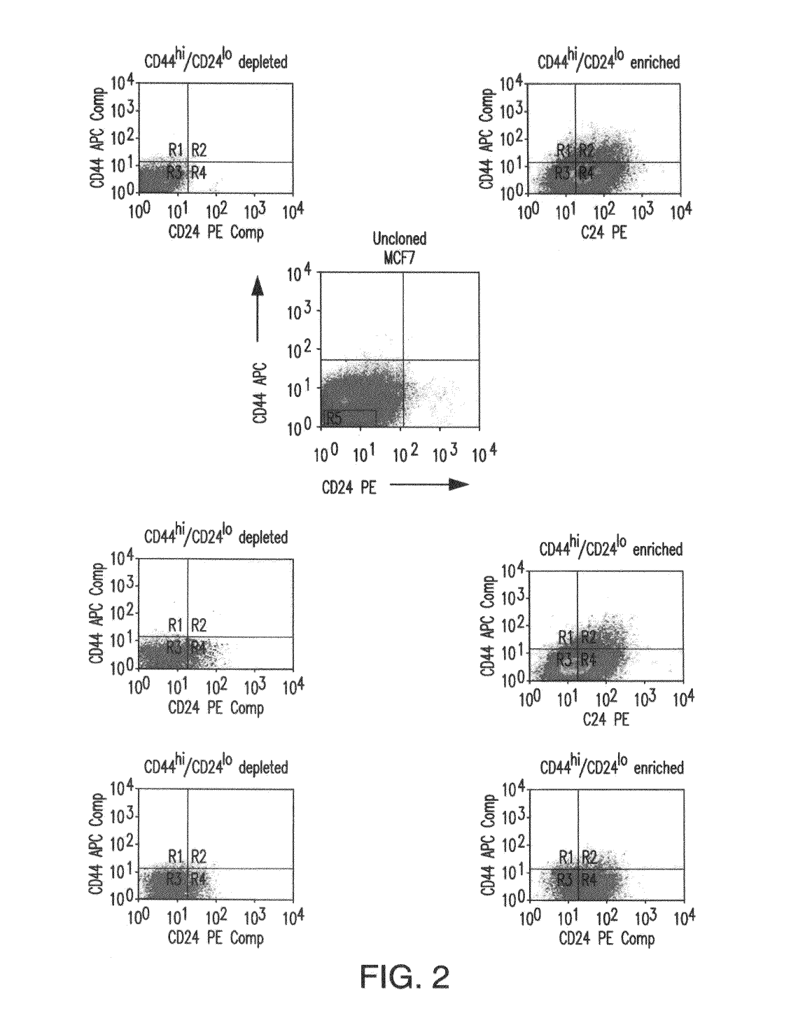

2. CD44hi/CD24lo CSCs rapidly differentiate. After expanding clonal populations from the above two groups, cell surface expression of CD44 and CD24 were evaluated. As seen in FIG. 2, CSC clones rapidly reverted to a phenotype closely resembling that of the unsorted MCF7 population. Therefore, as predicted, initially pure CSCs rapidly generate a more differentiated population comprised primarily of TACs or TAC-like cells (46).

3. GFP tagging of CSCs. A commonly used marker for CSCs, as well as for embryonal stem cells, is Oct3/4 a member of the POU family of transcription factors (24,39). In order to identify CSCs whose fates could be easily followed, unfractionated MCF7 cells were stably transfected with a plasmid encoding green fluorescent protein (GFP) under the control of a 4 kb segment of the human Oct3/4 proximal promoter. As expected, <1% of the resultant transfectants expressed GFP (FIG. 3A), in keeping with the results shown in FIG. 1A. Experiments were then performed to determine whether there was concordance between GFP expression and the CD44hi/CD24lo breast CSC phenotype. This was done in two ways. First, cell sorting was used to isolate GFP+ cells and then these cells were evaluated for CD44 and CD24 expression. As seen in FIG. 3B, virtually all of the GFP+ cells were CD44hi/CD24lo. The reciprocal experiment was also performed in which CD44hi/CD24lo CSCs were isolated and then assessed for expression of GFP. As seen in FIG. 3C, the entire CSC population also expressed GFP. Second, the morphologies and sizes of the two cell populations were evaluated. As seen in FIG. 4, CD44hi/CD24lo cells were distinctly smaller and rounder than the bulk of the MCF7 population.

4. Oct3/4-GFP+MCF7 cells remain “frozen” in a stem cell-like state. Because the Oct3/4-GFP+MCF7 population was concordant with the CD44hi/CD24lo CSC population, it had initially been expected that it would eventually differentiate into a TAC or TAC-like population as shown in FIG. 2. Thus, it had been predicted that these cells would not only lose their CD44hi/CD24lo phenotype but their expression of GFP as well due to a silencing of the Oct3/4 promoter as the cells differentiated into TACs. Surprisingly, it was found that both the cell surface phenotype and GFP positivity persisted. In fact, these phenotypes have remained stable after >20 wks of in vitro passage. Attempts have been made to force these cells to differentiate in vitro by a variety of methods including co-culturing them with MCF7 TACs but have been uniformly unsuccessful.

The persistence of the Oct3/4-GFP+MCF7 (and CD44hi/CD24lo) population suggested that these cells might in fact be true CSCs that for some reason had been “locked” or “frozen” in a CSC-like state. In order to further test this idea and to attempt another means of forcing the cells to differentiate into TACs, the capacity of the cells for de novo tumor initiation was tested. 10⁴ of these cells were inoculated subcutaneously in nude mice; concurrently 5×10⁶ unfractionated MCF7 cells were inoculated in a different group of animals. In the latter case, 3/3 animals developed tumors, which first became apparent 6-8 wks after inoculation. In contrast, 3/3 animals inoculated with the much smaller number of Oct3/4-GFP+ MCF7 cells (104/animal) developed tumors by 2 wks, consistent with the known high efficiency of CSCs purified by CD44/CD24 selection. Thus, Oct3/4-GFP+MCF7 cells were at least 500-fold better than their TAC counterparts at de novo tumor initiation, which is considered to be the “gold standard” for the CSC.

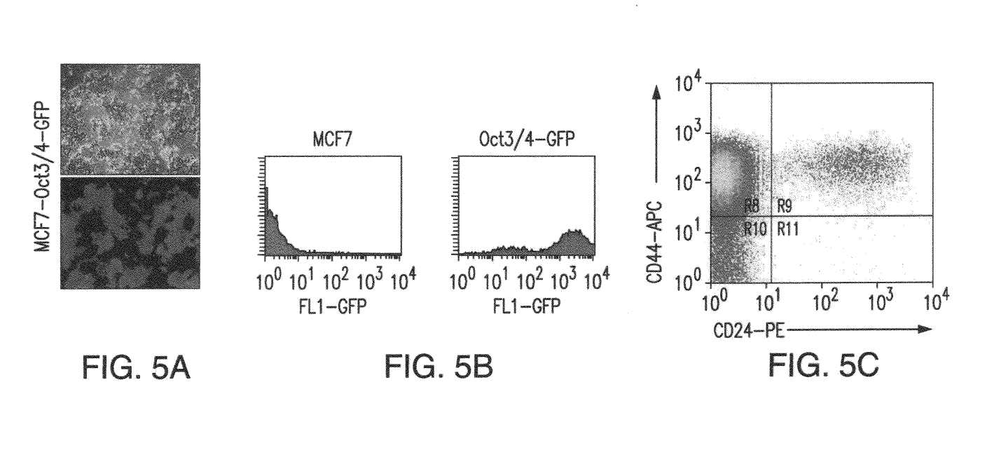

The tumor cell population arising from the initial inoculum of these Oct3/4-GFP+MCF7 cells was further examined. Single cell suspensions were prepared from the tumors, grown for two weeks in G418-containing medium to eliminate host cells, and again analyzed for CSC-like properties. The recovered tumor cells retained full expression of both GFP and the CSC phenotype (CD44hi/CD24lo: >95%) (FIG. 5).

In other studies, Oct3/4-GFP+MCF7 cells were examined by qRT-PCR for expression of the breast cancer CSC markers CD133 and Oct3/4. After normalizing to GAPDH, the ratios of these transcripts in Oct3/4-GFP+MCF7 cells and non-CSC-MCF7 cells were 6.3 for CD133 and 2.5 for Oct3/4. It was also asked whether Oct3/4-GFP+MCF7 cells and non-CSC-MCF7 cells showed differences in the so-called “side population” (SP) in which CSCs have been shown to reside (10,29,46). The SP is due to CSCs ability to exclude the dye Hoechst 33342 and reflects high expression of ABCG2 transporters such as MDR1 and BRCP (30). The foregoing results showed the SP fraction to comprise 6.3% of MCF7 cells and 23.8% of the Oct3/4-GFP+MCF7 cell population. These fractions could also be reduced by >70% following exposure to 20 μM Reserpine, which inhibits ABC transporters (30). Moreover, the overall mean fluorescence intensity of the entire Oct3/4-GFP+MCF population was two-fold lower than that of the MCF7 population. Thus, based on independent qRT-PCR assessments of CSC gene expression profiles and on uptake/exclusion of Hoechst 33342, one can conclude that the Oct3/4-GFP+MCF7 population consists of highly enriched and stable cells that are unable to differentiate into TACs.

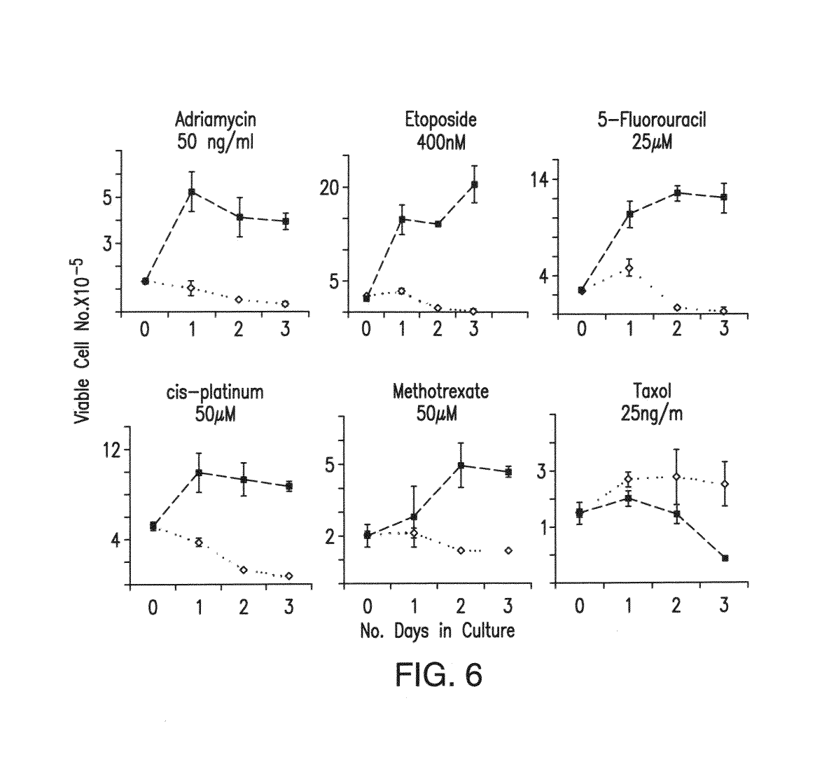

5. Differential chemosensitivities of Oct3/4-GFP+MCF7 cells. It is believed that CSCs are more resistant to chemotherapeutic agents than TACs, thus at least partly accounting for the propensity of many tumors to relapse after an initial response (12,27,36). To test this idea, MCF7 cells and Oct3/4-GFP+MCF7 CSCs were exposed to six different chemotherapeutic agents commonly employed to treat breast cancer. As seen in FIG. 6, Oct3/4-GFP+MCF7 cells showed significantly greater resistance in five of the six cases and, in fact, even increased in number over the course of the study. The exception to this was seen in the case of taxol where Oct3/4-GFP+MCF cells were significantly more sensitive than MCF7 cells.

### 7. WORKING EXAMPLE 2

A highly sensitive assay for compounds with selectivity for CSCs. The identification of compounds that selectively target Oct3/4-GFP+MCF7 cells demands a screening strategy that is simple, sensitive, rapid, and reproducible. Ideally, it should also be internally controlled to allow for the detection of what might initially be only small differences between the two cell populations. To this end, control MCF7 cells were tagged with the red fluorescent protein DsRED (Clontech). It was shown that the overall fluorescence intensity of this population as well as that of the Oct3/4-GFP+MCF7 CSCs varies in direct proportion to the number of initially plated cells. Further, GFP and DsRED fluorescence were simultaneously measured at non-overlapping emission/excitation wavelengths, and fractional changes of the two populations were reproducibly measured (FIG. 7). Such a labeled population may be used to identify compounds selectively inhibitory to Oct3/4-GFP+MCF7 cells by detecting reductions in the GFP:DsRED ratio. The assay has been adapted from a 96 well plate to a 384 well plate format without loss of sensitivity or specificity.

### 8. WORKING EXAMPLE 3

Experiments were performed to determine whether the growth of Oct3/4-GFP(+) MCF cells could be inhibited with novel small molecules. Oct3/4-GFP(+) MCF7 cells were used as the target population. Control cells consisted of Oct3/4-GFP(−)MCF cells (lacking an Oct3/4-GFP cassette) that were infected with a GFP-expressing lentivirus as a way of controlling for GFP expression. Initial screenings were performed in 384 well plates on triplicate samples as follows.

First, the cell cultures were prepared by trypsinizing and resuspending the cells in MCF7 culture medium (alpha MEM, 10% FBS, 2 mM Glutamine, 1 mM Sodium Pyruvate, 1 mM non-essential amino acids, 100 U/ml Penicillin/Streptomycin) at a density of 4×10⁴/ml. The screen was then performed by (1) diluting compounds to 30 μM using alpha MEM; (2) adding 5 p. 1 of compound-containing solution to each well (which will result in a final concentration of 5 μM); (3) adding 25 μl of cell suspension to each well (1000 cells/well); and (4) incubating the cells with compounds for 72 hours at 37° C., 5% CO₂. Cell viability was then assessed using the CellTiter-Blue® assay by Promega, modified as follows. Since there were 30 ul medium in each well, 6 μl CellTiter-Blue was added to each well. The plates were then incubated at 37° C. for 2.5 hours. The plates were then read on a SpectraMax M5 Microplate Reader using the wavelength of excitation 560 nm/emission 590 nm.

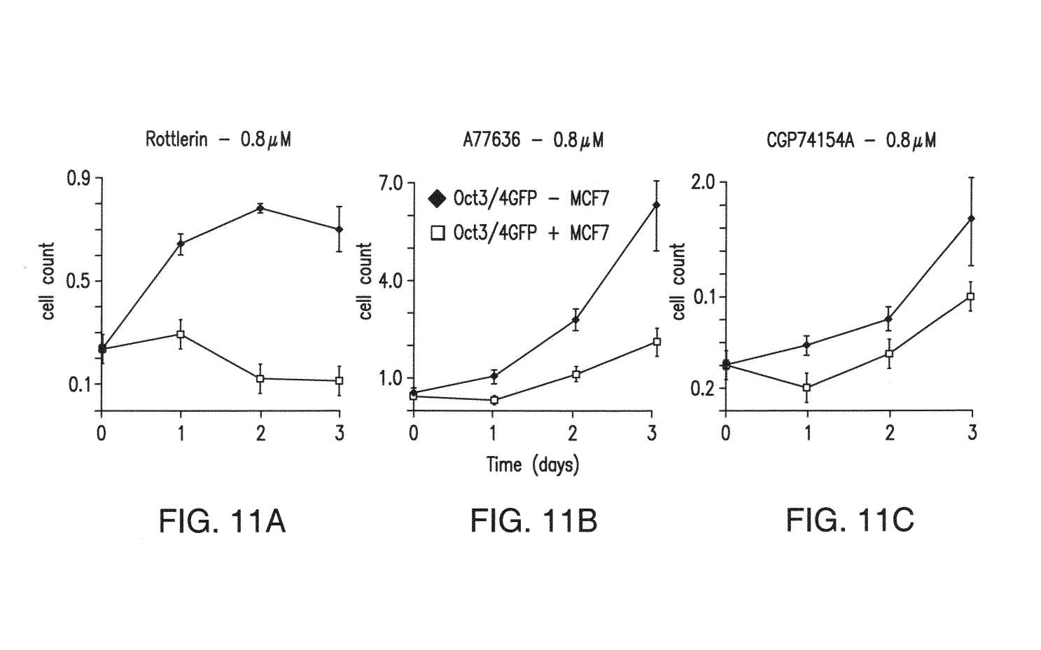

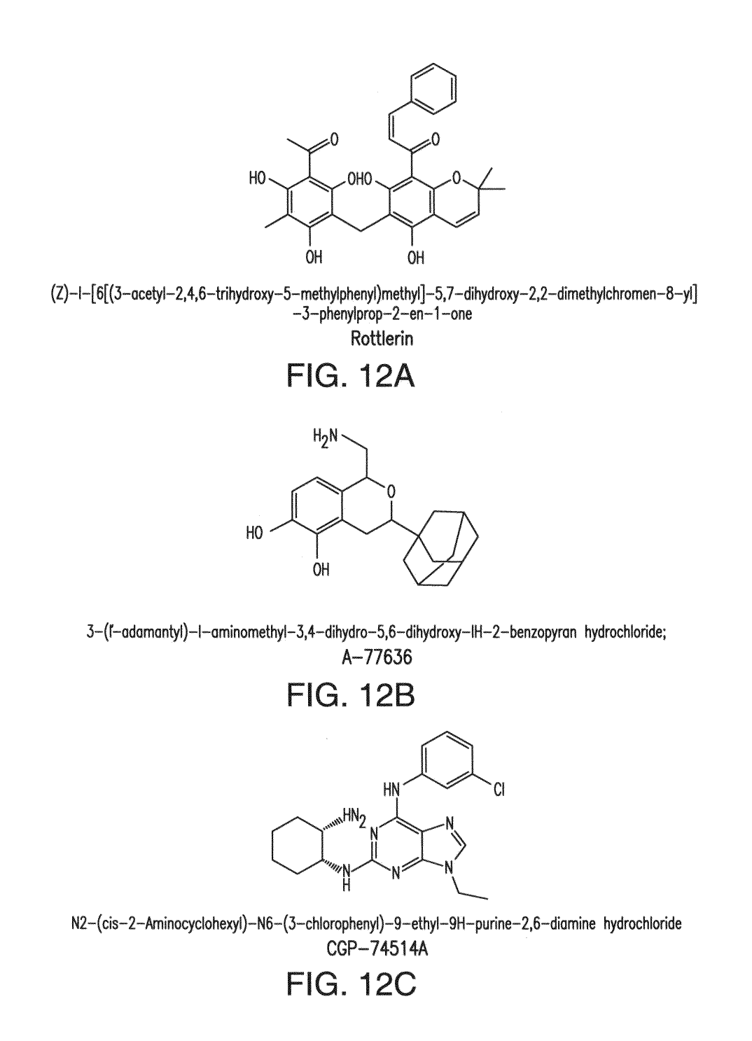

After identifying Rottlerin, (Z)-1-[6-[(3-acetyl-2,4,6-trihydroxy-5-methylphenyl)methyl]-5,7-dihydroxy-2,2-dimethylchromen-8-yl]-3-phenylpro-2-en-1-one, a protein kinase inhibitor having the structure shown in FIG. 12A (Gschwendt et al., 1994, Biochem. Biophy. Res. Comm. 199(1):93-98); A77636, 3-(1′-adamantyl)-1-aminomethyl-3,4-dihydro-5,6-dihydroxy-1H-2-benzopyran hydrochloride, a dopamine receptor agonist having a structure shown in FIG. 12B (Ryman-Rasmussen et al., 2007, Neuropharmacol. 52(2):562-575) and CGP74514A, N2-(cis-2-aminocyclohexyl)-N6-(3-chlorophenyl)-9-ethyl-9H-purine-2,6-diamine hydrochloride, a CDK-1 specific kinase inhibitor having a structure shown in FIG. 12C, as potentially selective agents, each compound was re-tested at several concentrations for its ability to inhibit each cell line. Then, a secondary set of experiments were performed in 12 well plates in which 2000 cells/well were initially seeded and allowed to attach for one day. The following day (day 0), the medium was replaced with fresh medium containing 0.8 μM of each of these three compounds. Cell counts were then performed manually on triplicate wells over a period of three days. Oct3/4-GFP+ MCF7 cells are represented by the lower, dashed curve in all three panels. The results are shown in FIGS. 11A, B and C

### 9. WORKING EXAMPLE 4

A lentivirus vector (“lentivector”) was constructed for introduction of a nucleic acid comprising an Oct3/4 promoter sequence linked to nucleic acid encoding GFP (see sequence set forth in FIG. 9A-B) into cells. The vector was constructed using two lentiviral vectors provided by Dr. Curt Civin at Johns Hopkins, one of which contained the EF1a promoter and the other of which contained a lentiviral backbone with a Ubc9-GFP transcription cassette. In the latter, the GFP coding sequence was replaced with a Neo resistance cassette to form a lenti-Ubc9-Neo vector, into which the EF1a promoter was inserted. The result is the construct shown in FIG. 13. To produce an Oct3/4-GFP expression vector, the ca. 4.7 kb Oct3/4-GFP fragment was cloned into the HpaI site of this vector and then the EF1a promoter fragment was excised to produce the vector pLentiNeo-Oct3/4-GFP. The pLentiNeo-Oct3/4-GFP vector was observed to infect cells very well, and the percent of GFP+ cells following infection was found to be somewhat higher than the percent obtained after transfection, but the cells were observed to behave similarly.

### 10. WORKING EXAMPLE 5

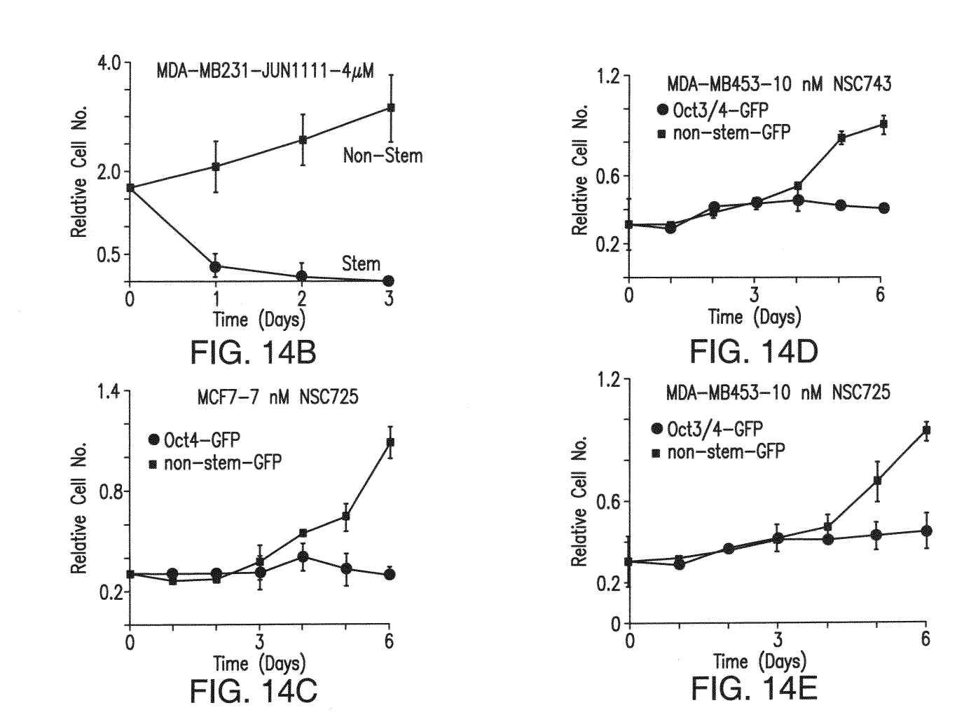

1280 compounds from the Library of Pharmacologically Active Compounds (“LOPAC”) were screened for activity against non-stem cell and Oct3/4-GFP+ CSCs prepared from three breast cancer cell lines, MCF7, MB231 and MB453 using methods set forth above. As shown in FIG. 14A, β-lapachone, rottlerin, and A77636 selectively inhibited growth of CSCs from a tleast one of these cell lines. FIG. 14B-D shows CSC-selective inhibitory activity of compounds JUN1111 (at 4 μM; FIG. 14B), NSC725 (at 7 nM and 10 nM: FIGS. 14C and E, respectively) and NSC&43 (at 10 nM; FIG. 14D).

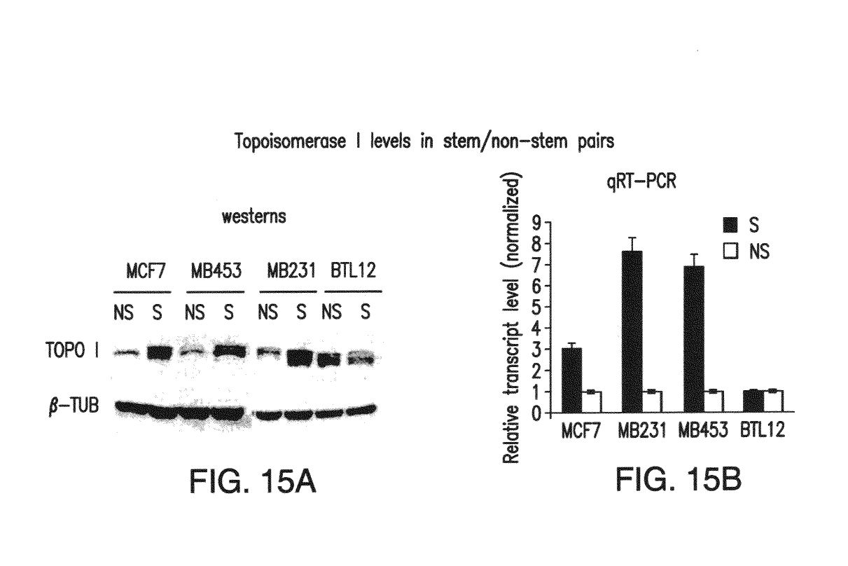

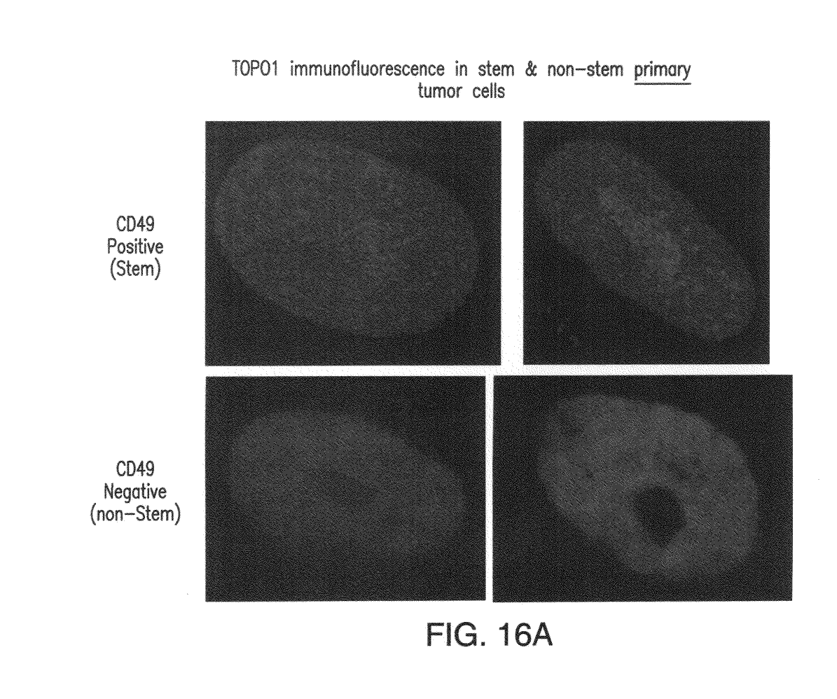

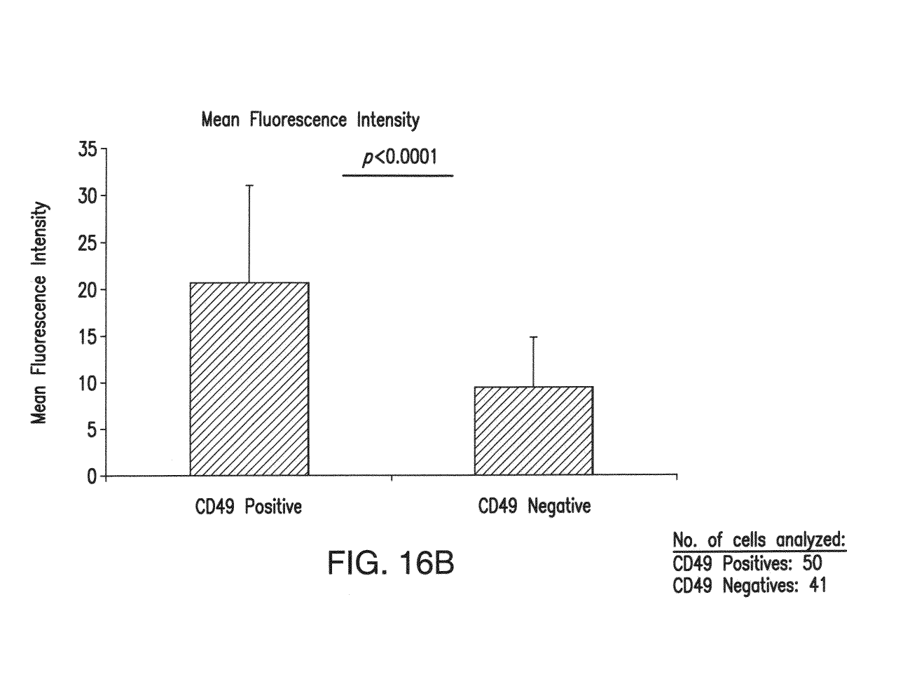

β-lapachone, NSC-743 and NSC-725 have activity as topoisomerase I inhibitors. Accordingly, as these three compounds all showed CSC-selective inhibitory activity, the possibility that topoisomerase I activity could be a distinguishing feature of CSCs was explored. Indeed, as shown in the western blot depicted in FIG. 15A the level of topoisomerase I protein and its corresponding mRNA transcript (FIG. 15B) was increased in the CSC versions of MCF7, MDA-MB453 and MDA-MB231 cells, although not in CSCs prepared from BTL12 cells. These results were supported by immunofluorescence studies of primary tumor cells using antibodies directed against topoisomerase I (Sigma-Aldrich Cat. No HPA019039 Rabbit anti-human TopoI) which showed substantially greater binding to CD49(+) CSC than to CD49 (−) non-stem cells (FIG. 16) The cells were isolated by FACS with CD49f mAb and were stained only with anti-TopoI (red) and DAPI (a nuclear stain-blue).

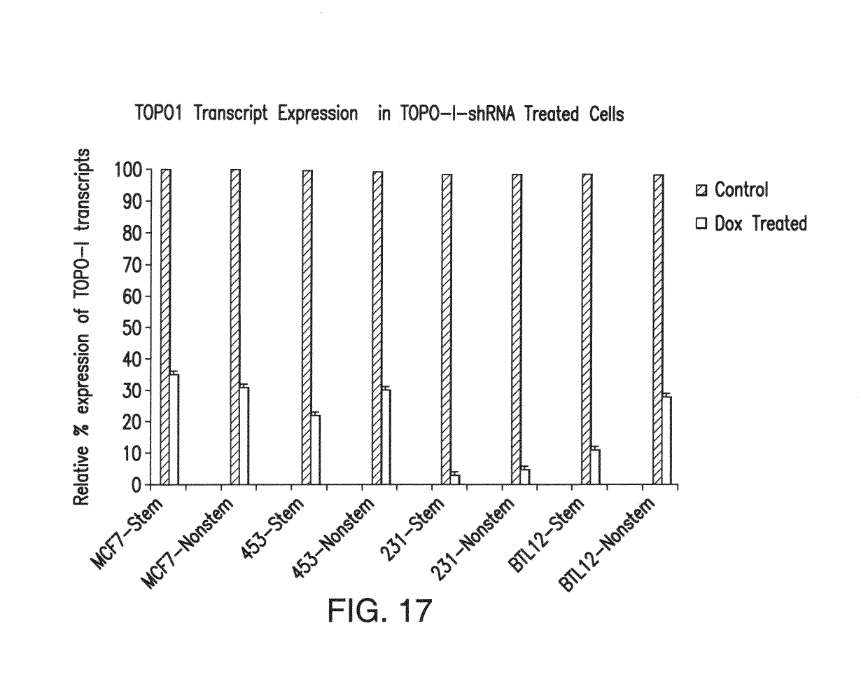

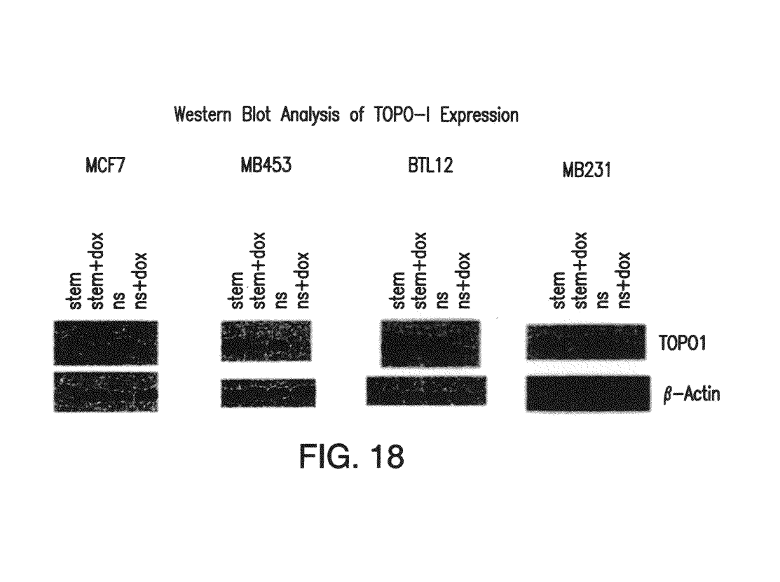

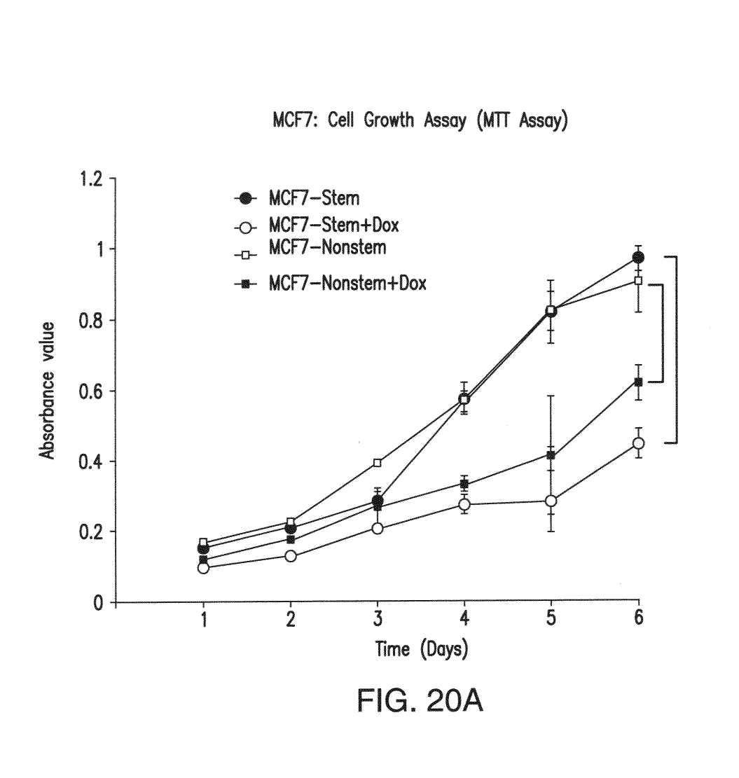

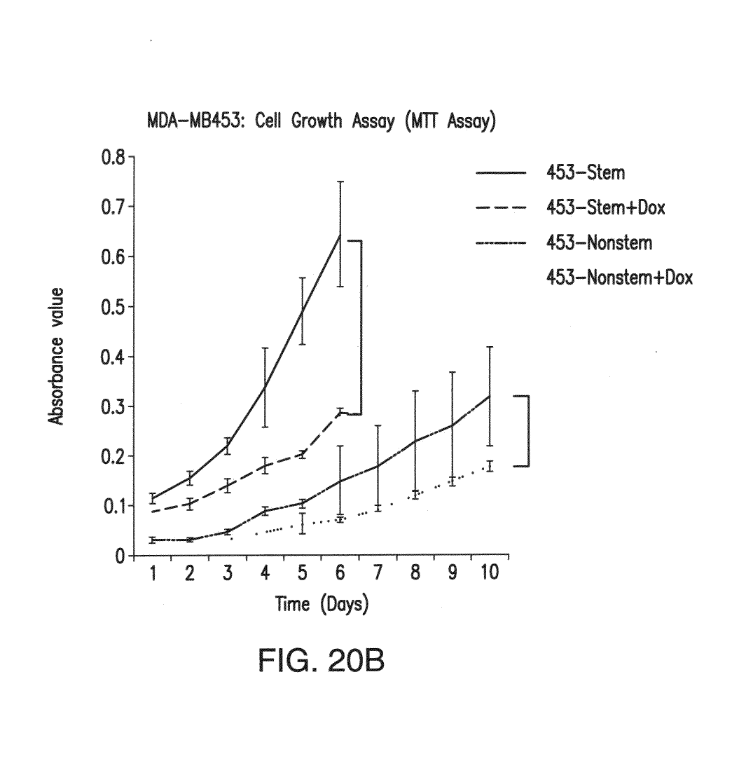

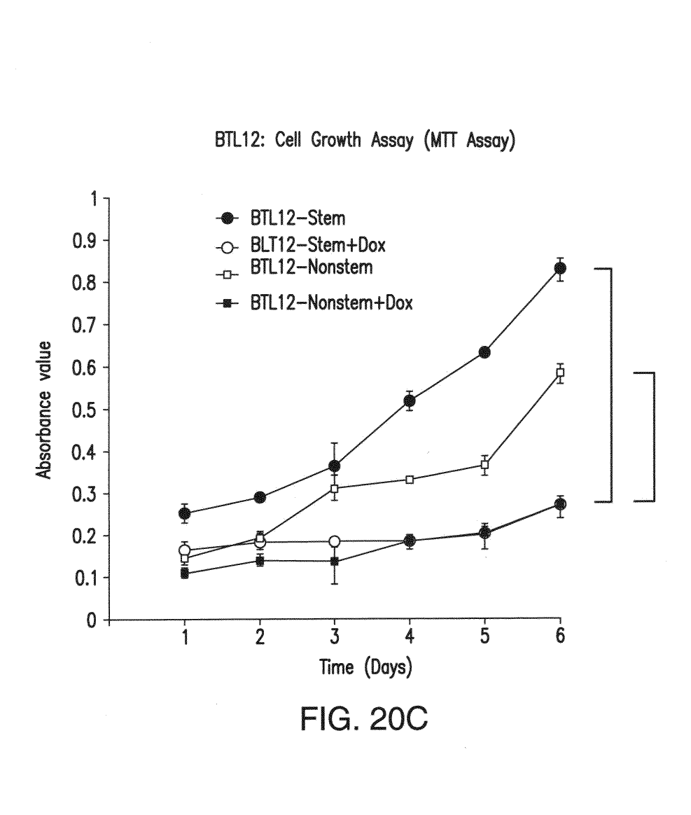

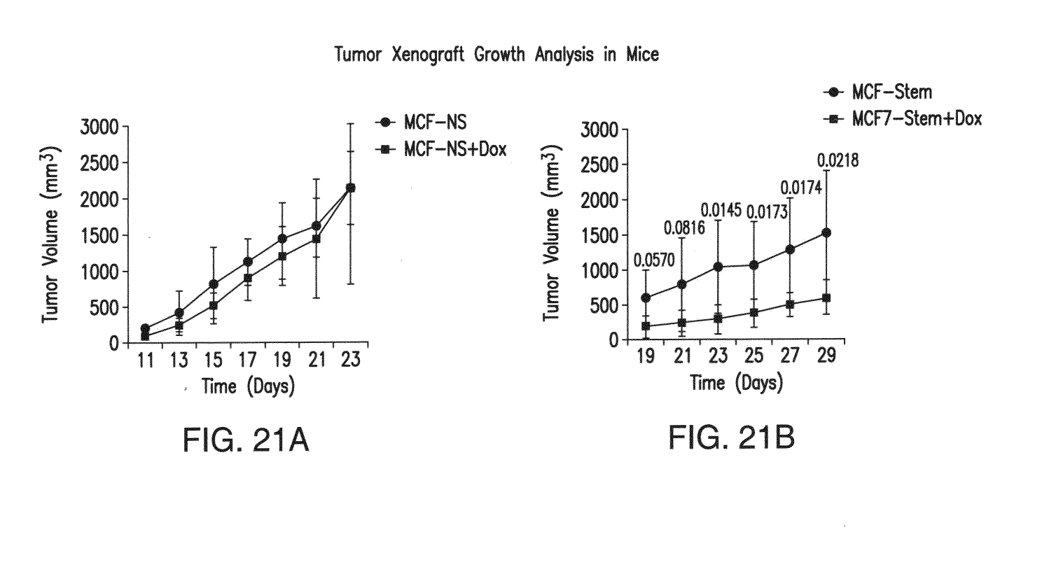

In view of the above results, topoisomerase I was considered a presumptive target for agents that selectively inhibit CSC growth. To test this hypothesis further, small hairpin RNA (shRNA) was used to knockdown topoisomerase I using a conditional shRNA expression system (pTRIPZ vector-Origene, Inc.), in which expression of the shRNA is induced by doxycycline. The shRNA used had the sequence: TGCTGTTGACAGTGAGCGCGCTGATTATAAACCTAAGAAATAGTGAAGCCAC AGATGTATTTCTTAGGTTTATAATCAGCATGCCTACTGCCTCGGA (SEQ ID NO:10, NM_{—}003286). As shown in FIGS. 17 and 18, doxycycline induced expression of shRNA homologous to the topoisomerase I gene successfully knocked-down mRNA and protein expression, respectively When the effect of doxycycline-induced shRNA knockdown of topoisomerase I was tested in cultures of non-stem and CSC versions of MCF7, MDA-MB453 and BTL12 cells, in all cases the effect of knockdown was, proportionally speaking, more profound on CSCs relative to their non-stem cell counterparts, although for MDA-MB453 and BTL12 cells, the number of viable cells was lowest for the non-stem cells with topoisomerase knockdown (FIG. 20A-C). Similarly, in tumor xenografts resulting from injection of non-stem and CSC MCF7 cells into nude mice, while doxycycline-induced topoisomerase knockdown decreased the size of tumors resulting from both non-stem and CSC MCF7 cells, the reduction in tumor size was proportionally greater for the CSC-generated tumors (FIG. 21A-B).

### 11. WORKING EXAMPLE 6

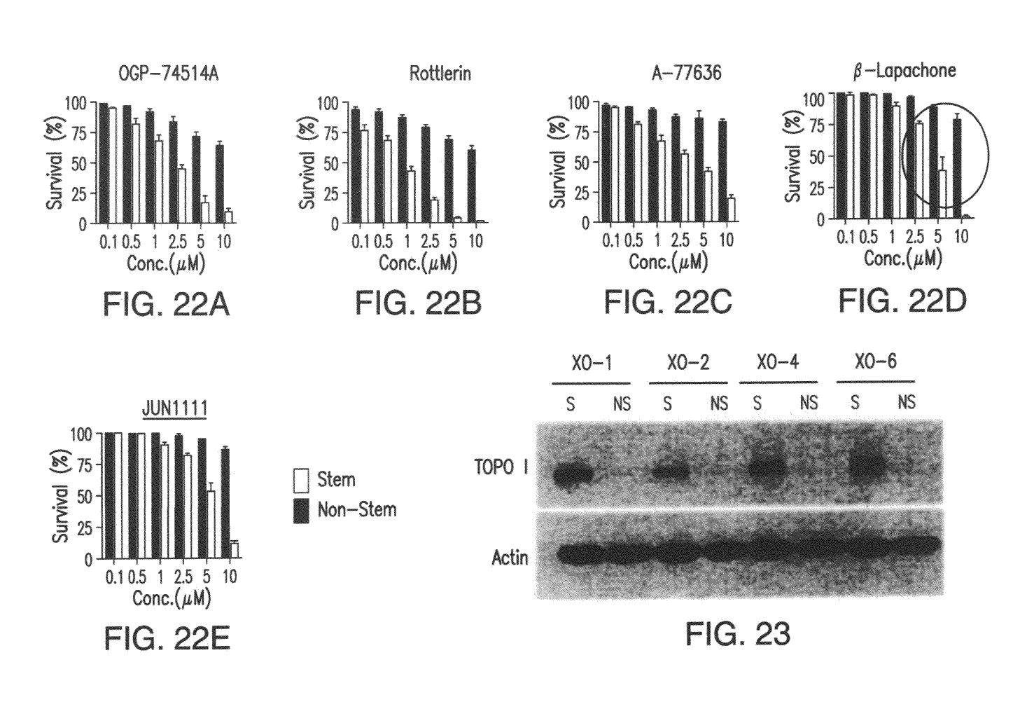

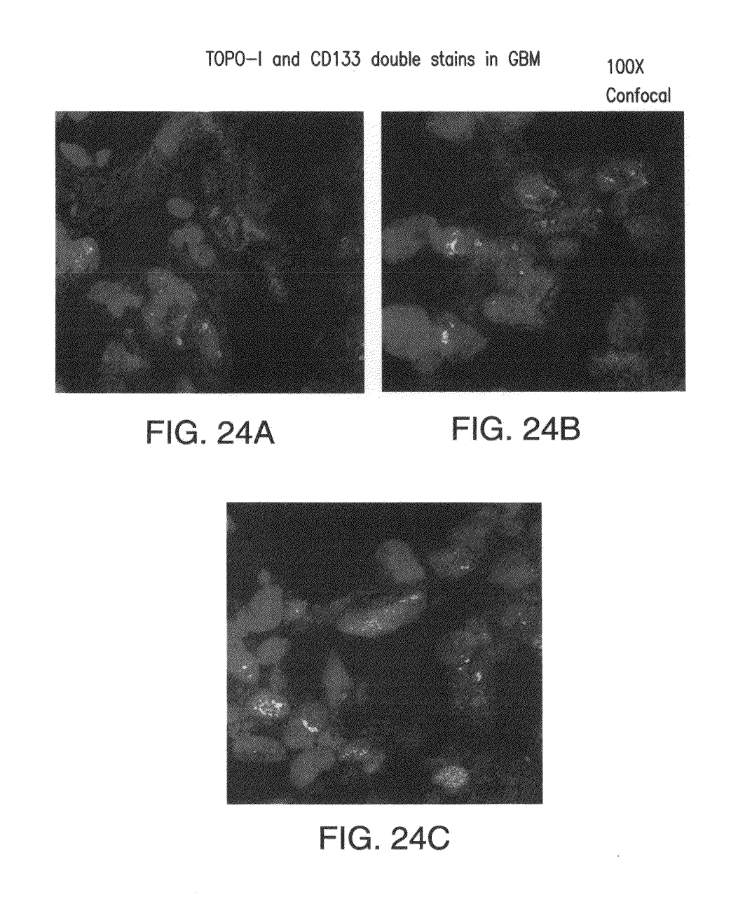

Because breast cancer cells and glioblastoma cells have been recognized as bearing some level of similarity to each other (Horvath et al., 2006, Proc. Natl. Acad. Sci. U.S.A. 103:17402-7), and in view of the results in the preceding section, levels of topoisomerase I were measured in glioblastoma cells by Western blot and immunofluorescence. FIG. 22A-E show the effects of CGP-74514A, rottlerin, A-77636, β-lapachone and Jun1111, respectively, on CSC (stem) and non-stem cells from a single primary glioblastoma tumor. FIG. 23 shows a Western blot showing expression of topoisomerase I in glioblastoma cells and their stem cell counterparts prepared from four different patients. FIG. 24A-C shows immunofluorescent staining of topoisomerase I (light blue green) and CD133 (red) in primary glioblastoma tumor cells from three individual patients, showing the close correlation between CD133 expression and Topol staining.

### 12. REFERENCES

1. Al-Hajj M, Wicha M S, Benito-Hernandez A, Morrison S J, Clarke MF. Prospective identification of tumorigenic breast cancer cells. Proc Natl. Acad Sci USA. 2003. 100: 3983-3988.
2. Bebek G, Yang J. PathFinder: mining signal transduction pathway segments from protein-protein interaction networks. BMC Bioinformatics. 2007. 8: 335-351.
3. Bernards R, Brummelkamp T R, Beijersbergen R L. shRNA libraries and their use in cancer genetics. Nat. Methods. 2006. 3: 701-706.
4. Brass A L, Dykxhoorn D M, Benita Y, Yan N, Engelman A, Xavier R J, Lieberman J, Elledge S J. Identification of host proteins required for HIV infection through a functional genomic screen. Science. 2008. 319: 921-926.
5. Brown M D, Gilmore P E, Hart C A, Samuel J D, Ramani V A, George N J, Clarke N R. Characterization of benign and malignant prostate epithelial Hoechst 33342 side populations. Prostate. 2007. 67:1384-13896.
6. Cao Y A, Wagers A J, Beilhack A, Dusich J, Bachmann M H, Negrin R S, Weissman I L, Contag C H. Shifting foci of hematopoiesis during reconstitution from single stem cells. Proc Natl Acad Sci USA. 2004. 101: 221-226.
7. Dalerba P, Cho R W, Clarke M F. Cancer stem cells: models and concepts. Annu Rev Med. 2007. 58: 267-284.
8. Dalerba P, Dylla S J, Park I K, Liu R, Wang X, Cho R W, Hoey T, Gurney A, Huang E H, Simeone D M, Shelton A A, Parmiani G, Castelli C, Clarke M F. Phenotypic characterization of human colorectal cancer stem cells. Proc Natl Acad Sci USA. 2007. 104: 10158-10163.
9. Fire A. RNA-triggered gene silencing. Trends Genet. 1999. 15: 358-363.
9a. Ginestier C, Hur M H, Charafe-Jauffret E, Monville F, Dutcher J, Brown M, Jacquemier J, Viens P, Kleer C G, Liu S, Schott A, Hayes D, Birnbaum D, Wicha M S, Dontu G. ALDH1 Is a marker of normal and malignant human mammary stem cells and a predictor of poor clinical outcome. Cell Stem Cell. 2007. 1: 555-567.
10. Hadnagy A, Gaboury L, Beaulieu R, Balicki D SP analysis may be used to identify cancer stem cell populations. Exp Cell Res. 2006. 312: 3701-3710.
11. Hurt E M, Kawasaki B T, Klarmann G J, Thomas S B, Farrar W L. CD44(+)CD24(−) prostate cells are early cancer progenitor/stem cells that provide a model for patients with poor prognosis. Br J. Cancer. 2008. 98:756-765.
12. Ieta K, Tanaka F, Haraguchi N, Kita Y, Sakashita H, Mimori K, Matsumoto T, Inoue H, Kuwano H, Mori M. Biological and genetic characteristics of tumor-initiating cells in colon cancer. Ann Surg Oncol. 2008.15: 638-648.
13. Johnson P H, Walker R P, Jones S W, Stephens K, Meurer J, Zajchowski D A, Luke M M, Eeckman F, Tan Y, Wong L, Parry G, Morgan T K Jr, McCarrick M A, Monforte J. Multiplex gene expression analysis for high-throughput drug discovery: screening and analysis of compounds affecting genes overexpressed in cancer cells. Mol Cancer Ther. 2002. 1: 1293-1304.
14. Jorgensen W L. The many roles of computation in drug discovery. Science. 2004. 303:1813-1818.
15. Kakarala M, Wicha M S. Cancer stem cells: implications for cancer treatment and prevention. Cancer J. 2007. 13: 271-275.
16. Lawson D A, Witte O N. Stem cells in prostate cancer initiation and progression. J Clin Invest. 2007. 117: 2044-2050.
17. Lee da Y, Gutmann D H. Cancer stem cells and brain tumors: uprooting the bad seeds. Expert Rev Anticancer Ther. 2007. 7: 1581-1590.
18. Li C, Heidt D G, Dalerba P, Burant C F, Zhang L, Adsay V, Wicha M, Clarke M F, Simeone D M. Identification of pancreatic cancer stem cells. Cancer Res. 2007. 67: 1030-1037.
19. Li Y, Lu J, Prochownik E V. c-Myc-mediated genomic instability proceeds via a megakaryocytic endomitosis pathway involving Gplbalpha. Proc Natl Acad Sci USA. 2007. 104: 3490-3495. Erratum in: Proc Natl Acad Sci USA. 2007. 104: 6491.
20. Liu R, Wang X, Chen G Y, Dalerba P, Gurney A, Hoey T, Sherlock G, Lewicki J, Shedden K, Clarke M F. The prognostic role of a gene signature from tumorigenic breast-cancer cells. N Engl J. Med. 2007. 356: 217-226.
21. Lobo N A, Shimono Y, Qian D, Clarke M F. The biology of cancer stem cells. Annu Rev Cell Dev Biol. 2007. 23: 675-699.
22. Martinez-Agosto J A, Mikkola H K, Hartenstein V, Banerjee U. The hematopoietic stem cell and its niche: a comparative view. Genes Dev. 2007. 21: 3044-3060.
23. Mello C C, Conte D Jr. Revealing the world of RNA interference. Nature. 2004. 431: 338-342.
24. Minucci S, Botquin V, Yeom Y I, Dey A, Sylvester I, Zand D J, Ohbo K, Ozato K, Scholer H R. Retinoic acid-mediated down-regulation of Oct3/4 coincides with the loss of promoter occupancy in vivo. EMBO J. 1996. 15: 888-899.
25. Morrison S J. The regulation of stem cell renewal. Presented at the AACR Special Conference in Cancer Research: The Role of Cancer Stem Cells in the Initiation and Propagation of Tumorigenesis. Feb. 12-15, 2008. Los Angeles, Calif.
26. North T E, Goessling W, Walkley C R, Lengerke C, Kopani K R, Lord A M, Weber G J, Bowman T V, Jang I H, Grosser T, Fitzgerald G A, Daley G Q, Orkin S H, Zon L I. Prostaglandin E2 regulates vertebrate haematopoietic stem cell homeostasis. Nature. 2007.447:1007-1011.
27. O'Brien C A, Pollett A, Gallinger S, Dick J E. A human colon cancer cell capable of initiating tumour growth in immunodeficient mice. Nature. 2007. 445:106-110.
28. Orkin S H, Zon L I. Hematopoiesis: an evolving paradigm for stem cell biology. Cell. 2008. 132: 631-644.
29. Patrawala L, Calhoun T, Schneider-Broussard R, Zhou J, Claypool K, Tang D G. Side population is enriched in tumorigenic, stem-like cancer cells, whereas ABCG2+ and ABCG2-cancer cells are similarly tumorigenic. Cancer Res. 2005. 65: 6207-6219.
30. Pearce H L, Safa A R, Bach N J, Winter M A, Cirtain M C, Beck W T. Essential features of the P-glycoprotein pharmacophore as defined by a series of reserpine analogs that modulate multidrug resistance. Proc Natl Acad Sci USA. 1989. 86: 5128-5132.
31. Ricci-Vitiani L, Lombardi D G, Pilozzi E, Biffoni M, Todaro M, Peschle C, De Maria R. Identification and expansion of human colon-cancer-initiating cells. Nature. 2007. 445:111-115.
32. Rothermund K, Rogulski K, Fernandes E, Whiting A, Sedivy J, Pu L, Prochownik E V.
C-Myc-independent restoration of multiple phenotypes by two C-Myc target genes with overlapping functions. Cancer Res. 2005. 65: 2097-2107.
33. Sánchez-García I, Vicente-Dueñas C, Cobaleda C. The theoretical basis of cancer-stem-cell-based therapeutics of cancer: can it be put into practice? Bioessays. 2007. 29: 1269-1280.
34. Sandy P, Ventura A, Jacks T. Mammalian RNAi: a practical guide. Biotechniques. 2005. 39: 215-224.
35. Schlabach M R, Luo J, Solimini N L, Hu G, Xu Q, Li M Z, Zhao Z, Smogorzewska A, Sowa M E, Ang X L, Westbrook T F, Liang A C, Chang K, Hackett J A, Harper J W, Hannon G J, Elledge S J. Cancer proliferation gene discovery through functional genomics. Science. 2008. 319: 620-624.
36. Shizuru J A, Negrin R S, Weissman I L. Hematopoietic stem and progenitor cells: clinical and preclinical regeneration of the hematolymphoid system. Arum Rev Med. 2005. 56: 509-538.
37. Silva J M, Marran K, Parker J S, Silva J, Golding M, Schlabach M R, Elledge S J, Hannon G J, Chang K. Profiling essential genes in human mammary cells by multiplex RNAi screening. Science. 2008. 319: 617-620.
38. Sussman R T, Ricci M S, Hart L S, Sun S Y, El-Deiry W S. Chemotherapy-resistant side-population of colon cancer cells has a higher sensitivity to TRAIL than the non-SP, a higher expression of c-Myc and TRAIL-receptor DR4. Cancer Biol Ther. 2007. 6:1490-1495.
39. Trosko J E. From adult stem cells to cancer stem cells: Oct-4 Gene, cell-cell communication, and hormones during tumor promotion. Ann N Y Acad. Sci. 2006. 1089: 36-58.
40. Wang H, Mannava S, Grachtchouk V, Zhuang D, Soengas M S, Gudkov A V, Prochownik E V, Nikiforov M A. c-Myc depletion inhibits proliferation of human tumor cells at various stages of the cell cycle. Oncogene. 2008. 27: 1905-1915.
41. Weisman J L, Liou A P, Shelat A A, Cohen F E, Guy R K, DeRisi J L. Searching for new antimalarial therapeutics amongst known drugs. Chem Biol Drug Des. 2006. 67:409-416.
42. Wicha M. personal communication
43. Wright M H, Calcagno A M, Salcido C D, Carlson M D, Ambudkar S V, Varticovski L. Brca1 breast tumors contain distinct CD44+/CD24− and CD133+ cells with cancer stem cell characteristics. Breast Cancer Res. 2008. February 1 [Epub ahead of print]
44. Xin L, Lawson D A, Witte O N. The Sca-1 cell surface marker enriches for a prostate-regenerating cell subpopulation that can initiate prostate tumorigenesis. Proc Natl Acad Sci USA. 2005.102: 6942-6947.
45. Yilmaz O H, Kiel M J, Morrison S J. SLAM family markers are conserved among hematopoietic stem cells from old and reconstituted mice and markedly increase their purity. Blood. 2006. 107: 924-930.
46. Zhou J, Wulflcuhle J, Zhang H, Gu P, Yang Y, Deng J, Margolick J B, Liotta L A, Petricoin E 3rd, Zhang Y. Activation of the PTEN/mTOR/STAT3 pathway in breast cancer stem-like cells is required for viability and maintenance. Proc Natl Acad Sci U S A. 2007. 104: 16158-16163.
47. Yin X, Giap C, Lazo J S, Prochownik E V. Low molecular weight inhibitors of Myc-Max interaction and function. Oncogene. 2003. 22: 6151-619.
48. Brideau C, Gunter B, Pikounis B, Liaw A. Improved statistical methods for hit selection in high-throughput screening. J Biomol Screen. 2003. 8: 634-647.
49. Zhijin Wu, Dongmei Liu, Yunxia Sui. Quantitative assessment of hit detection and confirmation in single and duplicate high-throughput screenings. J Biomol Screen. 2008. 13: 159-167.
50. Okamoto et al. Cell. 1990. 60:461-472.
51. Rosner et al. Nature. 1990. 345:686-692.
52. Sholer et al. Nature. 1990. 344:435-439.
53. Okamura et al., Dev. Biol. 2008. doi:10.1016/j.ydbio.2008.03.002.
54. Leendert et al. Cancer Res. 2003. 63:2244-2250.
55. Tai et al. Carcinogenesis. 2005. 26(2):495-502.
56. Palma et al. Cancer Letts. 2008. 263:204-211.
57. Hoei-Hansen. Cancer Treatment Rev. 2008. doi:10.1016/j.ctry.2007.12.007.
58. West et al. International Patent Application Publication No. WO 2007/019398 (Feb. 15, 2007).
59. Stanton et al. International Patent Application Publication No. WO 2004/080146 (Sep. 23, 2004).
60. Rana et al. International Patent Application Publication No. WO 2004/041838 (May 21, 2004).

Various publications are cited herein, the contents of which are hereby incorporated by reference in their entireties.

What is claimed is:

1. An isolated cancer stem cell containing an Oct3/4 promoter sequence which is not operably linked to an Oct 3/4 gene, wherein the cancer stem cell exhibits a stably undifferentiated cancer stem cell phenotype.

2. The cancer stem cell of claim 1, wherein said Oct3/4 promoter sequence is operably linked to a reporter gene and the reporter gene is detectably expressed.

3. An isolated nucleic acid comprising an Oct3/4 promoter sequence operably linked to a reporter gene, wherein the Oct3/4 promoter sequence does not contain the entire sequence set forth in FIG. 9 (GenBank Acc. No. DQ249177) (SEQ ID NO:1).

4. The nucleic acid of claim 3, comprised in a vector.

5. An isolated nucleic acid construct which may be used to reversibly immortalize a cancer stem cell, comprising an Oct3/4 promoter sequence operably linked to a reporter gene, wherein said promoter linked to the gene is flanked by lox sites.

6. The nucleic acid of claim 5, comprised in a vector.

7. A method of identifying an anti-cancer agent, comprising:
providing an isolated cancer stem cell containing an Oct3/4 promoter sequence which is not operably linked to an Oct3/4 gene;
(ii) providing a means for evaluating the proliferation, differentiation level, and/or viability of the cancer stem cell;
(iii) administering a test agent to the cancer stem cell; and
(iv) evaluating the proliferation and/or differentiation and/or viability of the cancer stem cell;
wherein an inhibition of proliferation, increase in level of differentiation, or decrease in viability associated with the presence of the test agent indicates that the test agent is an anti-cancer agent.

8. The method of claim 7 wherein the means for evaluating the proliferation, differentiation level, and/or viability comprises measuring and/or detecting expression of a reporter gene.

9. The method of claim 8, wherein the reporter gene encodes a fluorescent protein.

10. A method of identifying an anti-cancer agent with selective activity toward cancer stem cells, comprising:
(i) providing a population of cancer cells comprising cancer stem cells as well as cancer cells which are not stem cells, where the relative proportions of cancer stem cells and cancer cells which are not stem cells is known;
(ii) administering a test agent to the population of cells;
(iii) culturing the population after (ii); and
(iv) determining the relative proportions of cancer stem cells and cancer cells which are not stem cells in the population after (iii);
wherein a decrease in the relative proportion of cancer stem cells indicates that the test agent is an anti-cancer agent with selective activity against cancer stem cells.

11. The method of claim 10, further comprising providing a first means for detecting a cancer stem cell and a second, different means for detecting a cancer cell that is not a stem cell, where said first means and said second means are used to determine the relative proportions of cancer stem cells and cancer cells which are not stem cells.

12. The method of claim 11, wherein the first means and/or second means is a fluorescent antibody to a cell surface antigen.

13. The method of claim 11, wherein the first means and/or second means is an expression construct selectively expressed in a cancer stem cell or a cancer cell which is not a stem cell.

14. A method of identifying a gene associated with the cancer stem cell phenotype, comprising:
(i) providing an isolated cancer stem cell containing an Oct3/4 promoter sequence which is not operably linked to an Oct3/4 gene;
(ii) providing a means for evaluating the proliferation, differentiation level, and/or viability of the cancer stem cell;
(iii) administering a test interfering RNA to the cancer stem cell; and
(iv) evaluating the proliferation and/or differentiation and/or viability of the cancer stem cell;
wherein an inhibition of proliferation, increase in level of differentiation, or decrease in viability associated with the presence of the interfering RNA indicates that the test agent is an anti-cancer agent.

15. The method of claim 14 wherein the means for evaluating the proliferation, differentiation level, and/or viability comprises measuring and/or detecting expression of a reporter gene.

16. The method of claim 15, wherein the reporter gene encodes a fluorescent protein.

17. A means of identifying an agent likely to be of benefit to a subject, where the subject has a cancer, comprising:
(i) collecting a cancer stem cell from the subject;
(ii) introducing, into the cancer stem cell from the subject, a nucleic acid comprising as Oct3/4 promoter sequence operably linked to a reporter gene;
(iii) exposing the product of step (ii) to an agent; and
(iv) determining whether exposure to the agent inhibits proliferation, increases the level of differentiation, and/or decreases the viability of the product of step (ii),
where an inhibition of proliferation, increase in the level of differentiation, or decrease of viability indicates that the agent may be of therapeutic benefit to the subject.

18. A method for identifying an agent likely to be of benefit to a subject, where the subject has a cancer, comprising practicing the method of claim 17 for a number of different agents, and selecting the agent which most effectively, among those tested, inhibits proliferation, increases the level of differentiation, and/or decreases viability of the cancer stem cell containing the nucleic acid comprising an Oct3/4 promoter sequence operably linked to a reporter gene.
